SEQUENCE LISTING (26 sequences)

SEQ ID NO: 1 - skipped

SEQ ID NO: 2 - protein, 4 aa, synthetic construct
  REGION 1..4: note = SMA6
  SITE 2: note = Guanidino-L-alanine
  SITE 3: note = 2-indanyl-L-glycine
  SITE 1: note = N-terminal acetylation
  SITE 4: note = C-terminal 7-amino-4-carbamoylmethylcoumarin
KAGR

SEQ ID NO: 3 - protein, 4 aa, synthetic construct
  REGION 1..4: note = SMA7
  SITE 4: note = C-terminal 7-amino-4-carbamoylmethylcoumarin
  SITE 2: note = L-glutamic-acid-gamma-benzyl ester
  SITE 3: note = 2-indanyl-L-glycine
  SITE 1: note = N-terminal acetylation
KEGR

SEQ ID NO: 4 - skipped

SEQ ID NO: 5 - protein, 4 aa, synthetic construct
  REGION 1..4: note = SMA10
  SITE 2: note = Guanidino-L-alanine
  SITE 4: note = C-terminal 7-amino-4-carbamoylmethylcoumarin
  SITE 1: note = N-terminal acetylation
KAVR

SEQ ID NO: 6 - protein, 4 aa, synthetic construct
  REGION 1..4: note = SMA11
  SITE 1: note = N-terminal acetylation
  SITE 4: note = C-terminal 7-amino-4-carbamoylmethylcoumarin
  SITE 2: note = L-glutamic-acid-gamma-benzyl ester
KEVR

SEQ ID NO: 7 - skipped

SEQ ID NO: 8 - protein, 4 aa, synthetic construct
  REGION 1..4: note = SMA19
  SITE 2: note = L-glutamic-acid-gamma-benzyl ester
  SITE 1: note = N-terminal acetylation
  SITE 4: note = C-terminal 7-amino-4-carbamoylmethylcoumarin
KEKR

SEQ ID NO: 9 - protein, 4 aa, synthetic construct
  REGION 1..4: note = SMA25
  SITE 1: note = N-terminal acetylation
  SITE 4: note = C-terminal 7-amino-4-carbamoylmethylcoumarin
  SITE 2: note = L-glutamic acid allyl ester
  SITE 3: note = 2-indanyl-L-glycine
KEGR

SEQ ID NO: 10-13 - skipped (4 entries)

SEQ ID NO: 14 - protein, 4 aa, synthetic construct
  REGION 1..4: note = P-SMX152
  SITE 2: note = L-homoarginine
  SITE 3: note = 3-(1-naphthyl)-L-alanine
  SITE 4: note = C-terminal diphenyl phosphonate
  SITE 1: note = N-terminal Cy3 fluorophore attached by 6-aminohexanoic acid linker; note = D-proline
PRAR

SEQ ID NO: 15 - protein, 4 aa, synthetic construct
  REGION 1..4: note = I-SMX15
  SITE 1: note = N-terminal acetylation; note = D-proline
  SITE 3: note = 3-(1-naphthyl)-L-alanine
  SITE 4: note = C-terminal diphenyl phosphonate
  SITE 2: note = L-homoarginine
PRAR

SEQ ID NO: 16 - protein, 4 aa, synthetic construct
  SITE 1: note = N-terminal bodipy fluorophore attached by PEG linker; note = 2,6-dichlorobenzyl-L-tyrosine
  REGION 1..4: note = P-SMXI52
  SITE 2: note = Nle
  SITE 3: note = L-glutamic acid benzyl ester
  SITE 4: note = C-terminal diphenyl phosphonate
YLER

SEQ ID NO: 17 - protein, 4 aa, synthetic construct
  REGION 1..4: note = I-SMXI5
  SITE 1: note = N-terminal acetylation; note = 2,6-dichlorobenzyl-L-tyrosine
  SITE 4: note = C-terminal diphenyl phosphonate
  SITE 2: note = Nle
  SITE 3: note = L-glutamic acid benzyl ester
YLER

SEQ ID NO: 18 - skipped

SEQ ID NO: 19 - protein, 4 aa, synthetic construct
  REGION 1..4: note = Synthetic compound
  SITE 2: note = Guanidino-L-alanine
  SITE 3: note = 2-indanyl-L-glycine
KAGR

SEQ ID NO: 20 - protein, 4 aa, synthetic construct
  REGION 1..4: note = Synthetic compound
  SITE 2: note = L-glutamic-acid-gamma-benzyl ester
  SITE 3: note = 2-indanyl-L-glycine
KEGR

SEQ ID NO: 21 - skipped

SEQ ID NO: 22 - protein, 4 aa, synthetic construct
  REGION 1..4: note = Synthetic compound
  SITE 2: note = Guanidino-L-alanine
KAVR

SEQ ID NO: 23 - protein, 4 aa, synthetic construct
  REGION 1..4: note = Synthetic compound
  SITE 2: note = L-glutamic-acid-gamma-benzyl ester
KEVR

SEQ ID NO: 24 - skipped

SEQ ID NO: 25 - protein, 4 aa, synthetic construct
  REGION 1..4: note = Synthetic compound
  SITE 2: note = L-glutamic-acid-gamma-benzyl ester
KEKR

SEQ ID NO: 26 - protein, 4 aa, synthetic construct
  REGION 1..4: note = Synthetic compound
  SITE 2: note = L-glutamic acid allyl ester
  SITE 3: note = 2-indanyl-L-glycine
KEGR

(19) United States
(12) Patent Application Publication
(10) Pub. No.: US 2025/0208137 A1
(43) Pub. Date: Jun. 26, 2025

(54) COMPOUNDS FOR THE DETECTION AND INHIBITION OF COAGULATION PROTEASES

(71) Applicants: Politechnika Wroclawska, Wroclaw (PL); Cambridge Enterprise Limited, Cambridge (GB)

(72) Inventors: Marcin Drag, Wroclaw (PL); Paulina Kasperkiewicz-Wasilewska, Wroclaw (PL); Sylwia Modrzycka, Wroclaw (PL); James Andrew Hungtington, Cambridge (GB)

(21) Appl. No.: 18/847,111

(22) PCT Filed: Mar. 17, 2023
(86) PCT No.: PCT/GB23/50659

§ 371 Date: Sep. 13, 2024

(30) Foreign Application Priority Data
Mar. 17, 2022 (PL) P.440675

(51) Int. Cl. G01N 33/573; C07K 5/10

(52) U.S. Cl. CPC G01N 33/573; C07K 5/10; C07K 2319/60; G01N 2333/96433; G01N 2333/96444; G01N 2333/96452

(57) ABSTRACT

The present invention relates to novel compounds suitable for the detection and/or inhibition of coagulation proteases, specifically APC, fIIa, fXa, and fXIa. The compounds have the structural formula 1 or X shown below:

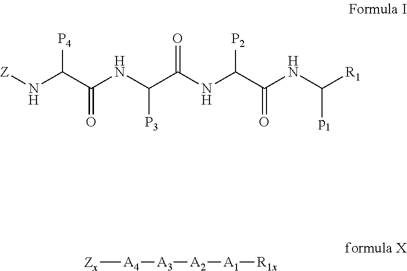

in which R₁, P₁, P₂, P₃, P₄, Z, R₁ₓ, A₁, A₂, A₃, A₄ and Zₓ are defined herein.

The present invention also relates to compositions comprising the compounds of formula I or formula X defined herein, to processes for synthesising these compounds and to their use for the treatment and/or detection of diseases and conditions in which coagulation proteases, in particular APC, fIIa, fXa, and fXIa, are implicated.

### INTRODUCTION

The present invention relates to novel compounds suitable for the detection and inhibition of coagulation proteases. More specifically, the present invention relates to novel compounds that are capable of detecting and/or inhibiting the coagulation proteases activated protein C (APC), thrombin (fIIa), fXa and fXIa (where f stands for “factor”). The present invention also relates to compositions comprising the compounds defined herein, to processes for synthesising these compounds, and to their use for the detection, evaluation and/or treatment of diseases and conditions in which coagulation proteases, in particular APC, fIIa, fXa, and fXIa, are implicated.

### BACKGROUND OF THE INVENTION

Hemostasis is a dynamic process for maintenance of a closed circulation system in which a damaged blood vessel is repaired by a fibrin-rich platelet plug to slow down, minimize, and eventually stop the bleeding [1]. The coagulation cascade is an intricate process involving several reactions in which serine proteases that circulate through the bloodstream as inactive zymogens are activated and next act as a catalyst to cleave yet more coagulation factors [2]. APC, fIIa, fXa, and fXIa are key players in the process of keeping the balance between coagulation and circulation of blood [3-5]. Dysregulation of their activity may lead to thrombotic diseases, a leading cause of preventable morbidity and mortality in developing and developed countries responsible for about 18 million deaths worldwide each year [6, 7]. Disturbance of natural occurring coagulation factors levels in plasma has been related to such life-threatening and debilitating blood diseases as hemophilia [8, 9], thrombosis [10-13], disseminated intravascular coagulation [14], ischemic stroke [15, 16], and myocardial infarction [17]. Abnormal coagulation protease activity has also been linked to cancer progression [18], Alzheimer's disease [19, 20], sepsis [21], and multiple sclerosis [22]. Additionally, recent studies have indicated the use of fIIa and fXa inhibitors in treating COVID-19 patients [23, 24]. All these reports about the presence of APC, fIIa, fXa, and fXIa in both physiological and pathophysiological states indicate the importance of monitoring their activity, as drug targets, and as potential diagnostic and prognostic markers.

Despite major progress in the understanding of coagulation system, the precise role of APC, fIIa, fXa, and fXIa in various blood clotting disorders and other diseases has not yet been established. This is mostly due to the complexity of the signaling pathways they participate in and the multitude of their interactions with each other, and with numerous protein molecules including other enzymes. Therefore, the object of the invention was to develop a set of potent and selective chemical tools for APC, fIIa, fXa, and fXIa to investigate their roles in coagulation and in other processes. In particular, the object of the invention is to provide selective substrates, inhibitors and activity based probes (ABP) of APC, fIIa, fXa, and fXIa.

### SUMMARY OF THE INVENTION

In one aspect, the present invention provides a compound of Formula I or X as defined herein, and/or a pharmaceutically acceptable salt, hydrate or solvate thereof.

In another aspect, the present invention provides a pharmaceutical composition which comprises a compound of Formula I or X as defined herein, or a pharmaceutically acceptable salt, hydrate or solvate thereof, and one or more pharmaceutically acceptable excipients.

In another aspect, the present invention provides a compound of Formula I or X as defined herein, or a pharmaceutically acceptable salt, hydrate or solvate thereof, or a pharmaceutical composition as defined herein, for use in therapy.

In another aspect, the present invention provides a compound of Formula I or X as defined herein, or a pharmaceutically acceptable salt, hydrate or solvate thereof, or a pharmaceutical composition as defined herein, for use in the treatment of a disease or condition in which coagulation proteases, in particular APC, fIIa, fXa, and fXIa, are implicated.

In another aspect, the present invention provides a compound of Formula I or X as defined herein, or a pharmaceutically acceptable salt, hydrate or solvate thereof, or a pharmaceutical composition as defined herein, for use in the treatment of a disease or condition associated with aberrant activity of one or more coagulation proteases, in particular APC, fIIa, fXa, and fXIa.

In another aspect, the present invention provides a compound of Formula I or X as defined herein, or a pharmaceutically acceptable salt, hydrate or solvate thereof, or a pharmaceutical composition as defined herein, for use in the inhibition of blood coagulation, wherein said compound is acting as an inhibitor of one or more coagulation proteases selected from fIIa, fXa and fXIa.

In another aspect, the present invention provides a compound of Formula I or X as defined herein, or a pharmaceutically acceptable salt, hydrate or solvate thereof, or a pharmaceutical composition as defined herein, for use in the treatment of bleeding disorders (e.g. hemophilia), wherein the compound is an inhibitor of APC.

In another aspect, the present invention provides a compound of Formula I or X as defined herein, or a pharmaceutically acceptable salt, hydrate or solvate thereof, or a pharmaceutical composition as defined herein, for use in the treatment of an illness connected with coagulation protease activity, wherein said compound is acting as an inhibitor of one or more coagulation proteases selected from APC, fIIa, fXa and fXIa.

In another aspect, the present invention provides the use of a compound of Formula I or X as defined herein, or a pharmaceutically acceptable salt, hydrate or solvate thereof, or a pharmaceutical composition as defined herein, in the manufacture of a medicament for use in the treatment of a disease or condition in which coagulation proteases, in particular APC, fIIa, fXa, and fXIa, are implicated.

In another aspect, the present invention provides the use of a compound of Formula I or X as defined herein, or a pharmaceutically acceptable salt, hydrate or solvate thereof, or a pharmaceutical composition as defined herein, in the manufacture of a medicament for use in the treatment of a disease or condition associated with activity of one or more coagulation proteases, in particular APC, fIIa, fXa, and fXIa.

In another aspect, the present invention provides the use of a compound of Formula I or X as defined herein, or a pharmaceutically acceptable salt, hydrate or solvate thereof, or a pharmaceutical composition as defined herein, in the manufacture of a medicament for use in the inhibition of blood coagulation, wherein said compound is acting as an inhibitor of one or more coagulation proteases selected from fIIa, fXa and fXIa.

In another aspect, the present invention provides the use of a compound of Formula I or X as defined herein, or a pharmaceutically acceptable salt, hydrate or solvate thereof, or a pharmaceutical composition as defined herein, in the manufacture of a medicament for use in the treatment of bleeding disorders (e.g. hemophilia), wherein the compound is an inhibitor of APC.

In another aspect, the present invention provides the use a compound of Formula I or X as defined herein, or a pharmaceutically acceptable salt, hydrate or solvate thereof, or a pharmaceutical composition as defined herein, in the manufacture of a medicament for use in the treatment of an illness connected with coagulation protease activity, wherein said compound is acting as an inhibitor of one or more coagulation proteases selected from APC, fIIa, fXa and fXIa.

In another aspect, the present invention provides a method of treating a disease or condition in which coagulation proteases, in particular APC, fIIa, fXa, and fXIa, are implicated, said method comprising administering to a subject in need thereof an effective amount of a compound of Formula I or X as defined herein, or a pharmaceutically acceptable salt, hydrate or solvate thereof, or a pharmaceutical composition as defined herein.

In another aspect, the present invention provides a method of treating a disease or condition associated with aberrant activity of one or more coagulation proteases, in particular APC, fIIa, fXa, and fXIa, said method comprising administering to a subject in need thereof an effective amount of a compound of Formula I or X as defined herein, or a pharmaceutically acceptable salt, hydrate or solvate thereof, or a pharmaceutical composition as defined herein.

In another aspect, the present invention provides a method of treating an illness connected with coagulation protease activity, said method comprising administering to a subject in need thereof an effective amount of a compound of Formula I or X as defined herein, or a pharmaceutically acceptable salt, hydrate or solvate thereof, or a pharmaceutical composition as defined herein.

In another aspect, the present invention provides a method of treating bleeding disorders (e.g. hemophilia), thrombosis, disseminated intravascular coagulation, ischemic stroke, myocardial infarction, cancer, Alzheimer's disease, sepsis, multiple sclerosis and/or COVID-19, said method comprising administering to a subject in need thereof an effective amount of a compound of Formula I or X as defined herein, or a pharmaceutically acceptable salt, hydrate or solvate thereof, or a pharmaceutical composition as defined herein.

In another aspect, the present invention provides a combination treatment comprising a compound of Formula I or X, or a pharmaceutically acceptable salt, hydrate or solvate thereof, as defined herein, with one or more additional therapeutic agents.

In another aspect, the present invention provides processes for preparing compounds of Formula I or X as defined herein, or a pharmaceutically acceptable salt, hydrate or solvate thereof, as defined herein, with one or more additional therapeutic agents.

Preferred, suitable, and optional features of any one particular aspect of the present invention are also preferred, suitable, and optional features of any other aspect.

### DETAILED DESCRIPTION OF THE INVENTION

### Definitions

Unless otherwise stated, the following terms used in the specification and claims have the following meanings set out below.

It is to be appreciated that references to “treating” or “treatment” include prophylaxis as well as the alleviation of established symptoms of a condition. “Treating” or “treatment” of a state, disorder or condition therefore includes: (1) preventing or delaying the appearance of clinical symptoms of the state, disorder or condition developing in a human that may be afflicted with or predisposed to the state, disorder or condition but does not yet experience or display clinical or subclinical symptoms of the state, disorder or condition, (2) inhibiting the state, disorder or condition, i.e., arresting, reducing or delaying the development of the disease or a relapse thereof (in case of maintenance treatment) or at least one clinical or subclinical symptom thereof, or (3) relieving or attenuating the disease, i.e., causing regression of the state, disorder or condition or at least one of its clinical or subclinical symptoms.

A “therapeutically effective amount” means the amount of a compound that, when administered to a mammal for treating a disease, is sufficient to effect such treatment for the disease. The “therapeutically effective amount” will vary depending on the compound, the disease and its severity and the age, weight, etc., of the mammal to be treated.

The compounds and intermediates described herein may be named according to either the IUPAC (International Union for Pure and Applied Chemistry) or CAS (Chemical Abstracts Service) nomenclature systems. It should be understood that unless expressly stated to the contrary, the terms “compounds of Formula I or X”, “compounds of the invention” and the more general term “compounds” refer to and include any and all compounds described by and/or with reference to Formula I or X herein. It should also be understood that these terms encompasses all stereoisomers, i.e. cis and trans isomers, as well as optical isomers, i.e. R and S enantiomers, of such compounds, in substantially pure form and/or any mixtures of the foregoing in any ratio. This understanding extends to pharmaceutical compositions and methods of treatment that employ or comprise one or more compounds of the Formula I or X, either by themselves or in combination with additional agents.

A wavy bond ( [symbol] ) is used herein to show a point of attachment.

The phrase “compound of the invention” means those compounds which are disclosed herein, both generically and specifically.

As used herein by itself or in conjunction with another term or terms, “pharmaceutically acceptable” refers to materials that are generally chemically and/or physically compatible with other ingredients (such as, for example, with reference to a formulation), and/or are generally physiologically compatible with the recipient (such as, for example, a subject) thereof.

As used herein by themselves or in conjunction with another term or terms, “subject(s)” and “patient(s)”, suitably refer to mammals, in particular humans.

### Compounds of the Invention

The first aspect of the invention provides a compound represented by the following chemical Formula I:

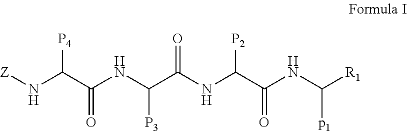

wherein:
Z is acetyl (Ac) or group of formula R₃-R₂-
   wherein: R₂ is linker group such as: PEG or 6-aminohexanoic acid; and
   R₃ is fluorescent group such as: 1-(5-carboxypentyl)-3,3-dimethyl-2-((1E,3Z)-3-(1,3,3-trimethylindolin-2-ylidene) prop-1-enyl)-3H-indolium (Cyanine 3), 1-(5-carboxypentyl)-2-((1E,3E,5E)-5-(1,3-dimethylindolin-2-ylidene) penta-1,3-dienyl)-3,3-dimethyl-3H-indolium (Cyanine 5), 1-(5-carboxypentyl)-3,3-dimethyl-2-((E)-2-((E)-3-((Z)-2-(1,3,3-trimethylindolin-2-ylidene)ethylidene)cyclohex-1-enyl) vinyl)-3H-indolium (Cyanine 7), or 4,4-difluoro-5,7-dimethyl-4-bora-3a,4a-diaza-s-indacene-3-propionic acid (Bodipy)
R₁ is reactive binding group such as: diphenyl phosphonate and its derivatives
P₁ is functional group:

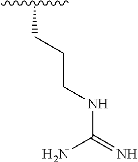

L-Arginine (L-Arg) side chain;
P₂ is a functional group selected from:

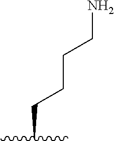

L-Lysine (L-Lys) side chain,

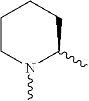

L-piperidine-2-carboxylic acid (L-Pip) side chain,

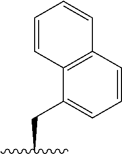

3-(1-naphthyl)-L-alanine (L-1-Nal) side chain,

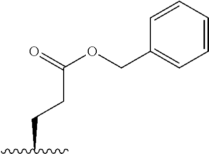

L-glutamic-acid-gamma-benzyl ester (L-Glu(Bzl)) side chain,
P₃ is functional group selected from:

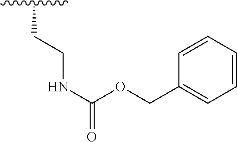

benzyloxycarbonyl-L-2,4-diaminobutyric acid (L-Dab(Z)) side chain,

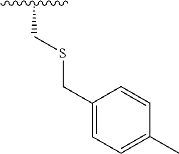

4-methylbenzyl-L-cysteine (L-Cys(MeBzl)) side chain,

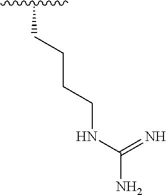

L-homoarginine (L-hArg) side chain,

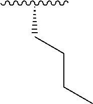

L-norleucine (L-Nle) side chain,
P₄ is functional group selected from:

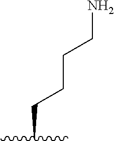

L-Lysine (L-Lys) side chain,

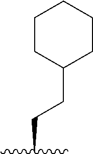

L-homocyclohexylalanine (L-hCha) side chain,

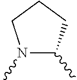

D-proline (D-Pro) side chain,

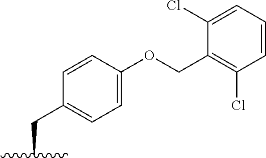

2,6-dichlorobenzyl-L-tyrosine (L-Tyr (2,6-Cl₂—Z) side chain.

Preferably, the compound according to the invention is characterized in that it has been selected from compounds represented by the following chemical formulas 2-5:

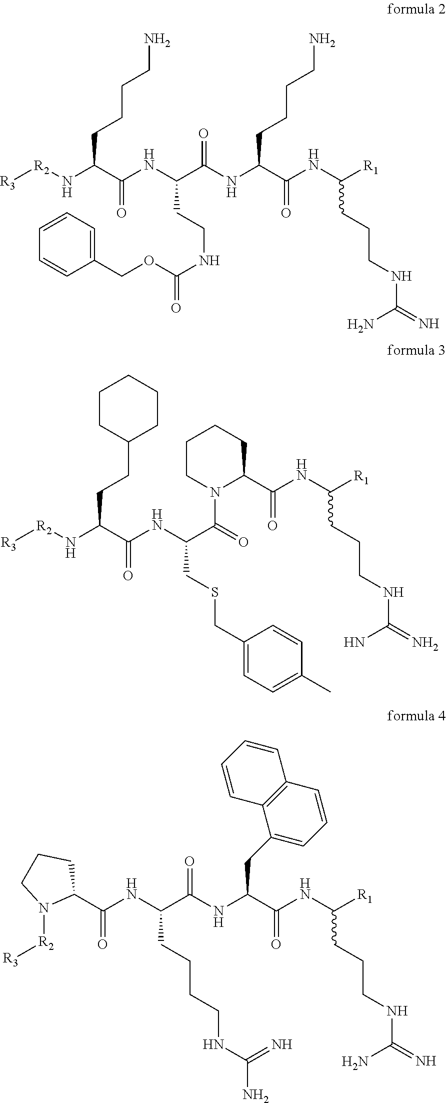

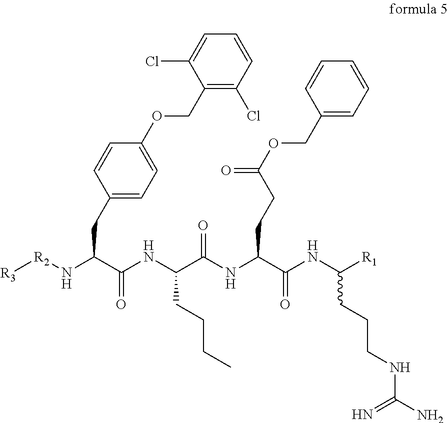

wherein:
R₃ is acetyl (Ac) or fluorescent group such as: 1-(5-carboxypentyl)-3,3-dimethyl-2-((1E,3Z)-3-(1,3,3-trimethylindolin-2-ylidene) prop-1-enyl)-3H-indolium (Cyanine 3), 1-(5-carboxypentyl)-2-((1E,3E,5E)-5-(1,3-dimethylindolin-2-ylidene) penta-1,3-dienyl)-3,3-dimethyl-3H-indolium (Cyanine 5), 1-(5-carboxypentyl)-3,3-dimethyl-2-((E)-2-((E)-3-((Z)-2-(1,3,3-trimethylindolin-2-ylidene)ethylidene)cyclohex-1-enyl) vinyl)-3H-indolium (Cyanine 7), or 4,4-difluoro-5,7-dimethyl-4-bora-3a,4a-diaza-s-indacene-3-propionic acid (Bodipy), R₂ is linker group such as: PEG or 6-aminohexanoic acid, R₁ is reactive binding group such as: diphenyl phosphonate and its derivatives.

Preferably, it has been selected from the following compounds:
Cy5-6-Ahx-Lys-Dab(Z)-Lys-Arg^{P}(OPh)₂,
Ac-Lys-Dab(Z)-Lys-Arg^{P}(OPh)₂,
Cy7-6-Ahx-hCha-Cys(MeBzl)-Pip-Arg^{P}(OPh)₂,
Ac-hCha-Cys(MeBzl)-Pip-Arg^{P}(OPh)₂,
Cy3-6-Ahx-D-Pro-hArg-1-Nal-Arg^{P}(OPh)₂,
Ac-DPro-hArg-1-Nal-Arg^{P}(OPh)₂,
Bodipy-PEG (4)-Tyr(2,6Cl₂—Z)-Nie-Glu(Bzl)-Arg^{P}(OPh)₂ and
Ac-Tyr(2,6Cl₂—Z)-Nle-Glu(Bzl)-Arg^{P}(OPh)₂
   which have been presented below:

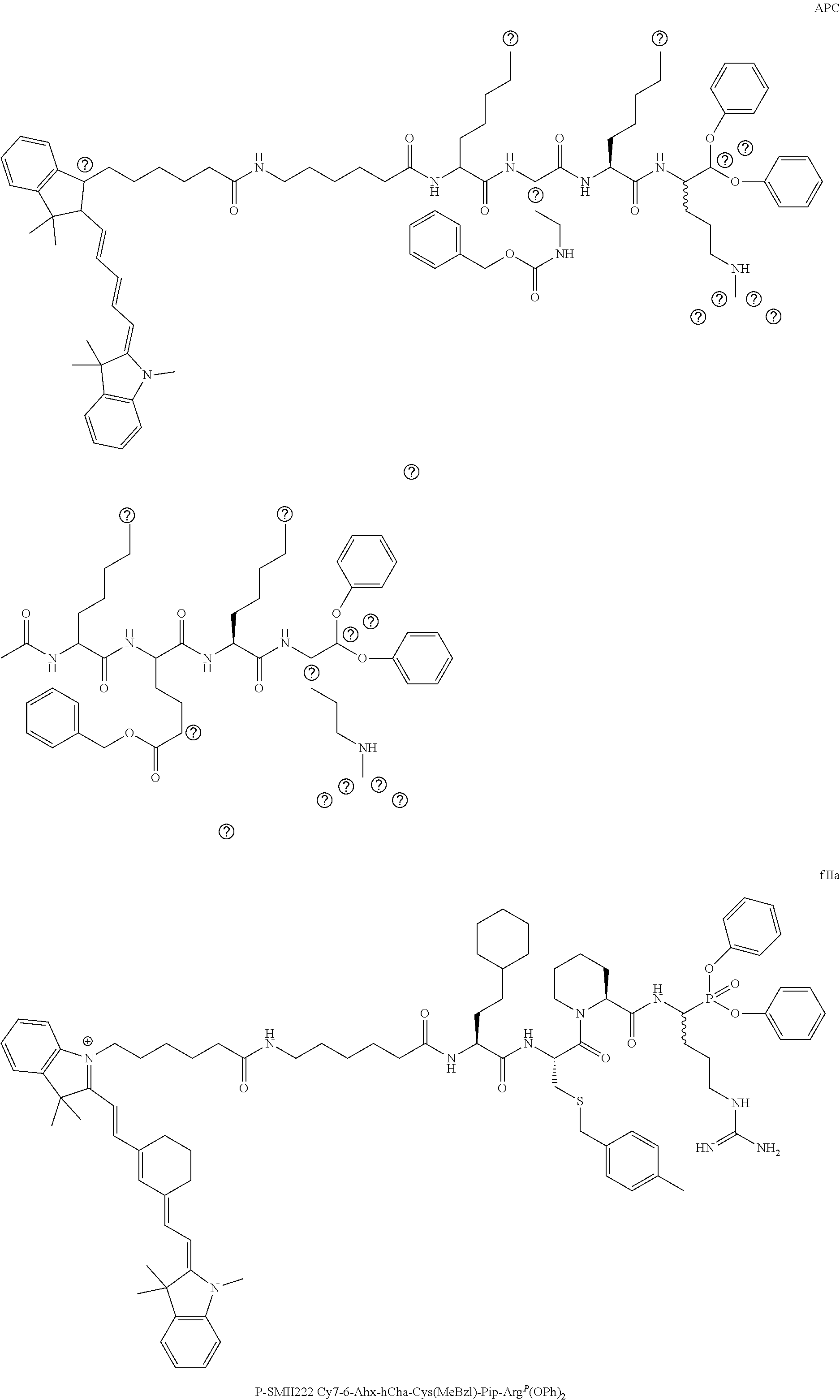

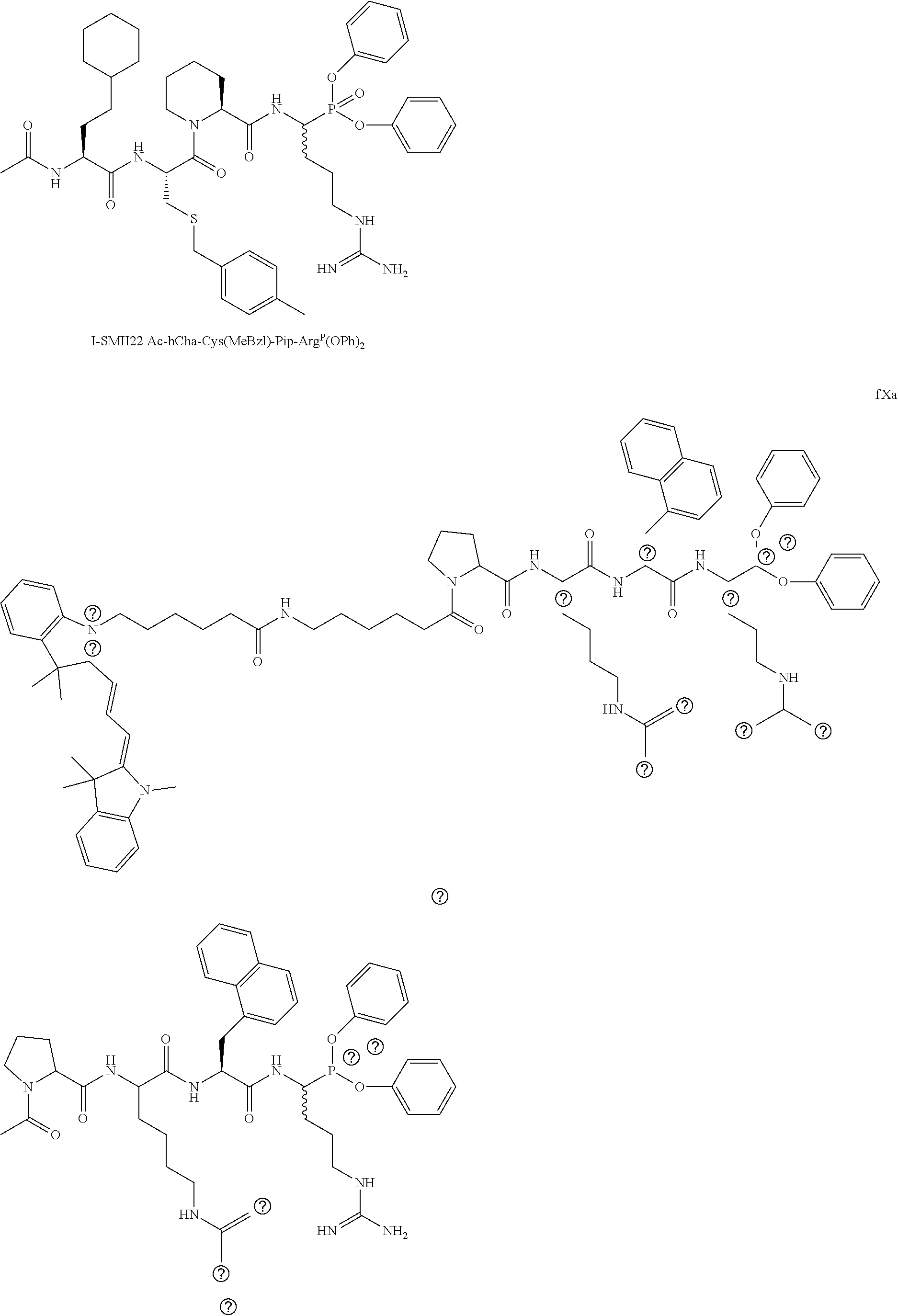

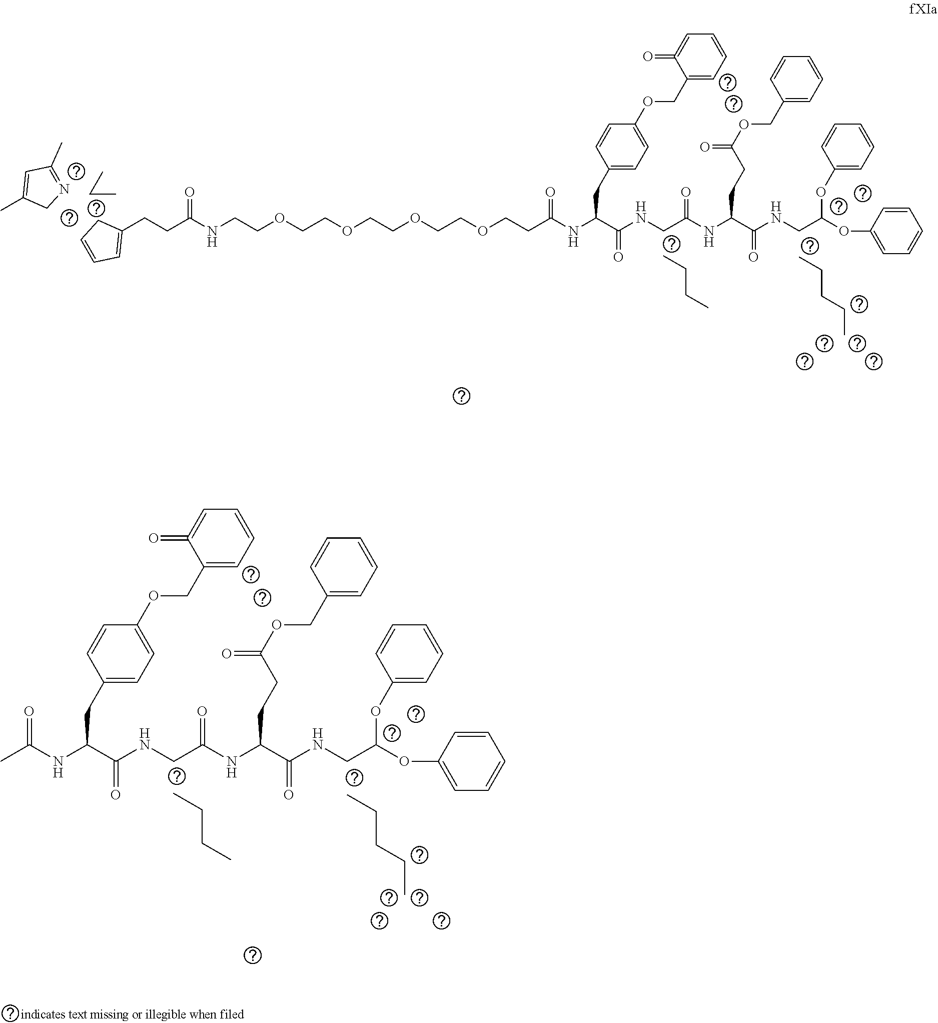

In a second aspect, the present invention provides a compound represented by the formula X shown below, or a pharmaceutically acceptable salt thereof:

Zₓ-A₄-A₃-A₂-A₁-R₁ₓ formula X
wherein:
Zₓ is hydrogen, an amino terminal capping group (e.g. (2-6C)alkanoyl, such as acetyl (Ac) or propionyl) or group of formula -R₂ₓ-R₃ₓ
   wherein:
      R₂ₓ is a linker group; and
      R₃ₓ is a fluorescent group or biotin;
R₁ₓ is a reactive binding group or a detectable leaving group;
A₁ is L-arginine (L-Arg);
A₂ is selected from
   L-lysine (L-Lys),
   L-piperidine-2-carboxylic acid (L-Pip),
   L-3-(1-naphthyl)-L-alanine (L-1-Nal),
   L-glutamic-acid-gamma-benzyl ester (L-Glu(Bzl)),
   L-glutamine (L-Gln),
   L-leucine (L-Leu),
   L-norleucine (L-Nle),
   L-threonine (L-Thr),
   L-valine (L-Val),
   L-homoarginine (L-hArg),
   4-amino-L-phenylalanine (L-Phe (4-NH₂)),
   L-homoalanine (L-Abu),
   L-phenylglycine (L-Phg),
   2-indanyl-L-glycine (L-IgI),
   L-neopentyl-glycine (L-NptGly), or
   L-norvaline (L-Nva);
As is selected from
   benzyloxycarbonyl-L-2,4-diaminobutyric acid (L-Dab(Z)),
   4-methylbenzyl-L-cysteine (L-Cys(MeBzl)),
   L-homoarginine (L-hArg),
   L-norleucine (L-Nle),
   D-Arginine (D-Arg),
   D-homophenylalanine (D-hPhe),
   guanidino-L-alanine (L-Agp),
   L-glutamic acid benzyl ester (LGlu(Bzl)),
   L-glutamic acid allyl ester (LGlu(All)),
   L-methionine sulfone (L-Met(O)₂), or
   L-biphenylalanine (L-Bip);
A₄ is selected from
   L-lysine (L-Lys),
   L-homocyclohexylalanine (L-hCha),
   D-proline (D-Pro),
   2,6-dichlorobenzyl-L-tyrosine (L-Tyr (2,6-Cl₂—Z),
   L-arginine (L-Arg),
   L-tryptophan (L-Trp),
   amino-L-homoalanine (L-Dab),
   N-trifluoroacetyl-L-lysine (L-Lys (TFA)),
   N-2-chlorobenzyloxycarbonyl-L-lysine (L-Lys (2-Cl—Z)),
   L-homoarginine (L-hArg),
   4-amino-L-phenylalanine (L-Phe (4-NH₂)),
   4-iodo-L-phenylalanine (L-Phe (4-I)),
   3-benzothienyl-L-alanine (L-Bta), or
   L-biphenylalanine (L-Bip).

In a third aspect, the present invention provides a compound represented by the formula X shown below, or a pharmaceutically acceptable salt thereof:

Zₓ-A₄-A₃-A₂-A₁-R₁ₓ formula X
wherein:
Zₓ is hydrogen, an amino terminal capping group (e.g. (2-6C)alkanoyl, such as acetyl (Ac) or propionyl) or group of formula -R₂ₓ-R₃ₓ
   wherein:
      R₂ₓ is a linker group; and
      R₃ₓ is a fluorescent group or biotin;
R₁ₓ is a reactive binding group or detectable leaving group;
A₁ is L-arginine (L-Arg);
A₂ is selected from
   L-lysine (L-Lys),
   L-glutamine (L-Gln),
   L-leucine (L-Leu),
   L-norleucine (L-Nle),
   L-threonine (L-Thr),
   L-valine (L-Val),
   L-homoarginine (L-hArg),
   4-amino-L-phenylalanine (L-Phe (4-NH₂)),
   L-homoalanine (L-Abu),
   L-phenylglycine (L-Phg),
   2-indanyl-L-glycine (L-IgI),
   L-neopentyl-glycine (L-NptGly), or
   L-norvaline (L-Nva);
A₃ is selected from
   benzyloxycarbonyl-L-2,4-diaminobutyric acid (L-Dab(Z)),
   D-Arginine (D-Arg),
   D-homophenylalanine (D-hPhe),
   guanidino-L-alanine (L-Agp),
   L-glutamic acid benzyl ester (LGlu(Bzl)),
   L-glutamic acid allyl ester (LGlu(All)),
   L-methionine sulfone (L-Met (O) 2), or
   L-biphenylalanine (L-Bip);
A₄ is selected from
   L-lysine (L-Lys),
   L-arginine (L-Arg),
   L-tryptophan (L-Trp),
   amino-L-homoalanine (L-Dab),
   N-trifluoroacetyl-L-lysine (L-Lys (TFA)),
   N-2-chlorobenzyloxycarbonyl-L-lysine (L-Lys (2-Cl—Z)),
   L-homoarginine (L-hArg),
   4-amino-L-phenylalanine (L-Phe (4-NH₂)),
   4-iodo-L-phenylalanine (L-Phe (4-I)),
   3-benzothienyl-L-alanine (L-Bta), or
   L-biphenylalanine (L-Bip).

The compounds of the third aspect defined above are particularly suited to targeting APC.

It will be appreciated that Zₓ may be any suitable amino terminal capping group known in the art. A skilled person would appreciate the kind of N-terminal moieties that are commonly used in the art. Suitably, Zₓ is a (2-6C)alkanoyl (e.g. acetyl or propionyl).

It will also be appreciated that R₂ₓ may be any suitable linker group that connects the fluorescent group R₃ₓ to the A₄ amino acid. Suitably, R₂ₓ may be a water soluble linker group. More suitably, R₂ₓ is selected from PEG or a group of the formula: —NH—(CH₂)₂₋₁₀C(O)OH (e.g. —NH—(CH₂)₅C(O)OH).

It will also be appreciated that R₃ₓ may be biotin or any suitable fluorescent group that can be used to detect the compound of formula X. The nature of the fluorescent group is not critical as long as it performs its function. Suitably, the fluorescent group is selected from one of the options outlined in paragraph (9) below.

It will also be appreciated that R₁ₓ may be any suitable detectable leaving group or reactive binding group. The nature of the leaving group or reactive binding group is not critical as long as it performs its function. Suitably, the reactive binding group is diphenyl phosphonate and its derivatives. Suitably, the detectable leaving group is selected from one of the options defined in paragraph (12) below.

Particular compounds of formula X herein include, for example, compounds of the formula X, or pharmaceutically acceptable salts, hydrates and/or solvates thereof, wherein, unless otherwise stated, each of Zₓ, R₂ₓ, R₃ₓ, R₁ₓ, A₂, A₃ and A₄ each have any of the meanings defined hereinbefore or are as defined in any one of paragraphs (1) to (25) hereinafter:—
(1) Zₓ is hydrogen, (2-4C)alkanoyl or group of formula -R₂ₓ-R₃ₓ;
(2) Zₓ is hydrogen, acetyl or propionyl or group of formula -R₂ₓ-R₃ₓ;
(3) Zₓ is acetyl or -R₂ₓ-R₃ₓ;
(4) R₂ₓ is a water soluble linker;
(5) R₂ₓ is a linker selected from: a PEG linker; or a linker of the formula:

*—NH—(CH₂)ₙ—C(O)—**
wherein * is the point of attachment to R₃ₓ and ** is the point of attachment to A₄ and n 1 to 10;
(6) R₂ₓ is a linker selected from: a PEG linker; or a linker of the formula:

*—NH—(CH₂)ₙ—C(O)—**
wherein * is the point of attachment to R₃ₓ and ** is the point of attachment to A₄ and n is 2 to 6;
(7) R₂ₓ is a linker selected from: a PEG linker; or a linker of the formula:

*—NH—(CH₂)ₙ—C(O)—**
wherein * is the point of attachment to R₃ₓ and ** is the point of attachment to A₄ and n is 2 to 6;
(8) R₂ₓ is a linker selected from: a PEG linker; or a linker of the formula:

*—NH—(CH₂)ₙ—C(O)—**
wherein * is the point of attachment to R₃ₓ and ** is the point of attachment to A₄ and n is 5;
(9) R₃ₓ is a fluorescent group selected from the group consisting of:
   Cyanine derivatives (e.g.:
      Cy2 (Benzoxazolium, 2-[3-[3-(5-carboxypentyl)-2 (3H)-benzoxazolylidene]-1-propen-1-yl]-3-ethyl-, inner salt),
      Cy3 (6-[(2E)-3,3-dimethyl-2-[(E)-3-(1,3,3-trimethylindol-1-ium-2-yl) prop-2-enylidene]indol-1-yl]hexanoic acid; chloride
      Cy5 (3H-Indolium, 2-[5-[1-(5-carboxypentyl)-1,3-dihydro-3,3-dimethyl-2H-indol-2-ylidene]-1,3-pentadien-1-yl]-1,3,3-trimethyl-, chloride),
      Cy7 (Sulfo-Cyanine7;2-[7-[1-(5-Carboxypentyl)-1,3-dihydro-3,3-dimethyl-5-sulfo-2H-indol-2-ylidene]-1,3,5-heptatrien-1-yl]-1-ethyl-3,3-dimethyl-5-sulfo-3H-indolium inner salt),
      Cy5.5 (Cy5.5 Carboxylic acid;2-(5-(3-(5-Carboxypentyl)-1,1-dimethyl-1,3-dihydro-2H-benzo[e]indol-2-ylidene) penta-1,3-dien-1-yl)-1,1,3-trimethyl-1H-benzo[e]indol-3-ium);
   Alexa Fluor series (e.g. AF488: 4-(6-amino-3-iminio-4,5-disulfonato-3H-xanthen-9-yl) isophthalate)
   ATTO series (e.g. ATTO 590 (2-(6,20-diethyl-7,7,9,17,19,19-hexamethyl-2-oxa-20-aza-6-azoniapentacyclo[12.8.0.0^{3,12}.0^{5,10}.0^{16,21}]docosa-1 (14),3,5,8,10,12,15,17,21-nonaen-13-yl)-4-(2,5-dioxopyrrolidin-1-yl)oxycarbonylbenzoic acid;perchlorate))
   Fluoresceine (3′,6′-dihydroxyspiro[isobenzofuran-1 (3H), 9′-[9H]xanthen]-3-one derivatives) and fluoresceine derivatives; and
   BODIPY (4,4-difluoro-5,7-dimethyl-4-bora-3a,4a-diaza-s-indacene-3-propionic acid).
(10) R₁ₓ is a reactive binding group selected from diphenyl phosphonate and its derivatives or a detectable leaving group selected from para-nitroaniline (pNA), 7-amino-4-carbamoylmethylcoumarin (ACC) and its derivatives, 7-amino-3-trifluoromethylcoumarin (AFC) and its derivatives, 7-amino-4-methylcoumarin (AMC (MCA)) and its derivatives, or rhodamine (3,6-diamino-9-(2-carboxyphenyl)-, chloride) and its derivatives;
(11) R₁ₓ is a reactive binding group selected from diphenyl phosphonate and its derivatives;
(12) R₁ₓ is a detectable leaving group selected from para-nitroaniline (pNA), 7-amino-4-carbamoylmethylcoumarin (ACC) and its derivatives, 7-amino-3-trifluoromethylcoumarin (AFC) and its derivatives, 7-amino-4-methylcoumarin (AMC (MCA)) and its derivatives, or rhodamine (3,6-diamino-9-(2-carboxyphenyl)-, chloride) and its derivatives;
(13) A₂ is selected from
   L-lysine (L-Lys),
   L-glutamine (L-Gln),
   L-leucine (L-Leu),
   L-norleucine (L-Nle),
   L-threonine (L-Thr),
   L-valine (L-Val),
   L-homoarginine (L-hArg),
   4-amino-L-phenylalanine (L-Phe (4-NH₂)),
   L-homoalanine (L-Abu),
   L-phenylglycine (L-Phg),
   2-indanyl-L-glycine (L-IgI),
   L-neopentyl-glycine (L-NptGly), or
   L-norvaline (L-Nva);
(14) A₂ is selected from
   L-lysine (L-Lys),
   L-valine (L-Val), or
   2-indanyl-L-glycine (L-IgI);
(15) A₂ is selected from
   L-lysine (L-Lys), or
   2-indanyl-L-glycine (L-IgI);
(16) A₂ is L-lysine (L-Lys);
(17) A₂ is L-valine (L-Val);
(18) A₂ is 2-indanyl-L-glycine (L-IgI);
(19) As is selected from
   benzyloxycarbonyl-L-2,4-diaminobutyric acid (L-Dab(Z)),
   D-Arginine (D-Arg),
   D-homophenylalanine (D-hPhe),
   guanidino-L-alanine (L-Agp),
   L-glutamic acid benzyl ester (LGlu(Bzl)),
   L-glutamic acid allyl ester (LGlu(All)),
   L-methionine sulfone (L-Met (O) 2), or
   L-biphenylalanine (L-Bip);
(20) As is selected from
   benzyloxycarbonyl-L-2,4-diaminobutyric acid (L-Dab(Z)),
   guanidino-L-alanine (L-Agp),
   L-glutamic acid benzyl ester (LGlu(Bzl)), or
   L-glutamic acid allyl ester (LGlu(All));
(21) A₃ is selected from
   benzyloxycarbonyl-L-2,4-diaminobutyric acid (L-Dab(Z)), or
   guanidino-L-alanine (L-Agp);
(22) A₃ is benzyloxycarbonyl-L-2,4-diaminobutyric acid (L-Dab(Z));
(23) A₃ is guanidino-L-alanine (L-Agp);
(24) A₄ is selected from
   L-lysine (L-Lys),
   L-arginine (L-Arg),
   L-tryptophan (L-Trp), or
   amino-L-homoalanine (L-Dab);
(25) A₄ is L-lysine (L-Lys).

In a particular group of compounds (group 1) of formula X:
A₁ is L-arginine (L-Arg); and
A₂ is as defined in paragraph (14) above;
and Zₓ, R₂ₓ, R₃ₓ, R₁ₓ A₃, and A₄ each have any one of the definitions set out herein.

In a particular group of compounds (group 2) of formula X:
A₁ is L-arginine (L-Arg); and
A₂ is as defined in paragraph (15) above;
and Zₓ, R₂ₓ, R₃ₓ, R₁ₓ A₃, and A₄ each have any one of the definitions set out herein.

In a particular group of compounds (group 3) of formula X:
A₁ is L-arginine (L-Arg); and
A₂ is as defined in paragraph (16) above;
and Zₓ, R₂ₓ, R₃ₓ, R₁ₓ A₃, and A₄ each have any one of the definitions set out herein.

In a particular group of compounds (group 4) of formula X:
A₁ is L-arginine (L-Arg); and
A₂ is as defined in paragraph (18) above;
and Zₓ, R₂ₓ, R₃ₓ, R₁ₓ A₃, and A₄ each have any one of the definitions set out herein.

In a particular group of compounds (group 5) of formula X:
A₁ is L-arginine (L-Arg); and
A₂ is as defined in paragraph (15) above; and
A₃ is as defined in paragraph (20) above;
and Zₓ, R₂ₓ, R₃ₓ, R₁ₓ and A₄ each have any one of the definitions set out herein.

In a particular group of compounds (group 6) of formula X:
A₁ is L-arginine (L-Arg); and
A₂ is as defined in paragraph (15) above; and
A₃ is as defined in paragraph (21) above;
and Zₓ, R₂ₓ, R₃ₓ, R₁ₓ and A₄ each have any one of the definitions set out herein.

In a particular group of compounds (group 7) of formula X:
A₁ is L-arginine (L-Arg); and
A₂ is as defined in paragraph (16) above; and
A₃ is as defined in paragraph (21) above;
and Zₓ, R₂ₓ, R₃ₓ, R₁ₓ and A₄ each have any one of the definitions set out herein.

In a particular group of compounds (group 8) of formula X:
A₁ is L-arginine (L-Arg); and
A₂ is as defined in paragraph (18) above; and
A₃ is as defined in paragraph (21) above;
and Zₓ, R₂ₓ, R₃ₓ, R₁ₓ and A₄ each have any one of the definitions set out herein.

In a particular group of compounds (group 9) of formula X:
A₁ is L-arginine (L-Arg); and
A₂ is as defined in paragraph (15) above;
A₃ is as defined in paragraph (21) above;
A₄ is as defined in paragraph (24) above; and
and Zₓ, Rex, R₃ₓ and R₁ₓ each have any one of the definitions set out herein.

In a particular group of compounds (group 10) of formula X:
A₁ is L-arginine (L-Arg); and
A₂ is as defined in paragraph (16) above; and
A₃ is as defined in paragraph (21) above;
A₄ is as defined in paragraph (24) above; and
and Zₓ, R₂ₓ, R₃ₓ and R₁ₓ each have any one of the definitions set out herein.

In a particular group of compounds (group 11) of formula X:
A₁ is L-arginine (L-Arg); and
A₂ is as defined in paragraph (18) above; and
A₃ is as defined in paragraph (21) above;
A₄ is as defined in paragraph (24) above; and
and Zₓ, R₂ₓ, R₃ₓ and R₁ₓ each have any one of the definitions set out herein.

In a particular group of compounds (group 12) of formula X:
A₁ is L-arginine (L-Arg); and
A₂ is as defined in paragraph (15) above;
A₃ is as defined in paragraph (21) above;
A₄ is as defined in paragraph (25) above; and
and Zₓ, R₂ₓ, R₃ₓ and R₁ₓ each have any one of the definitions set out herein.

In a particular group of compounds (group 13) of formula X:
A₁ is L-arginine (L-Arg); and
A₂ is as defined in paragraph (16) above; and
A₃ is as defined in paragraph (21) above;
A₄ is as defined in paragraph (25) above; and
and Zₓ, R₂ₓ, R₃ₓ and R₁ₓ each have any one of the definitions set out herein.

In a particular group of compounds (group 14) of formula X:
A₁ is L-arginine (L-Arg); and
A₂ is as defined in paragraph (18) above; and
A₃ is as defined in paragraph (21) above;
A₄ is as defined in paragraph (25) above; and
and Zₓ, R₂ₓ, R₃ₓ and R₁ₓ each have any one of the definitions set out herein.

Suitably, in any of the compound groups 1 to 14 above, either:
(i) Zₓ is as defined in paragraph (1) above, and/or
   R₂ₓ and R₃ₓ are as defined in paragraphs (4) and (9) respectively;
(ii) Zₓ is as defined in paragraph (1) above, and/or
   R₂ₓ and R₃ₓ are as defined in paragraphs (5) and (9) respectively;
(iii) Zₓ is as defined in paragraph (1) above, and/or
   R₂ₓ and R₃ₓ are as defined in paragraphs (6) and (9) respectively;
(iv) Zₓ is as defined in paragraph (1) above, and/or (iv)
   R₂ₓ and R₃ₓ are as defined in paragraphs (7) and (9) respectively;
(v) Zₓ is as defined in paragraph (1) above, and/or (v)
   R₂ₓ and R₃ₓ are as defined in paragraphs (8) and (9) respectively;
(vi) Zₓ is as defined in paragraph (2) above, and/or (vi)
   R₂ₓ and R₃ₓ are as defined in paragraphs (4) and (9) respectively;
(vii) Zₓ is as defined in paragraph (2) above, and/or
   R₂ₓ and R₃ₓ are as defined in paragraphs (5) and (9) respectively;
(viii) Zₓ is as defined in paragraph (2) above, and/or
   R₂ₓ and R₃ₓ are as defined in paragraphs (6) and (9) respectively;
(ix) Zₓ is as defined in paragraph (2) above, and/or
   R₂ₓ and R₃ₓ are as defined in paragraphs (7) and (9) respectively;
(x) Zₓ is as defined in paragraph (2) above, and/or
   R₂ₓ and R₃ₓ are as defined in paragraphs (8) and (9) respectively;
(xi) Zₓ is as defined in paragraph (3) above, and/or
   R₂ₓ and R₃ₓ are as defined in paragraphs (4) and (9) respectively;
(xii) Zₓ is as defined in paragraph (3) above, and/or
   R₂ₓ and R₃ₓ are as defined in paragraphs (5) and (9) respectively;
(xiii) Zₓ is as defined in paragraph (3) above, and/or
   R₂ₓ and R₃ₓ are as defined in paragraphs (6) and (9) respectively;
(xiv) Zₓ is as defined in paragraph (3) above, and/or
   R₂ₓ and R₃ₓ are as defined in paragraphs (7) and (9) respectively; or
(xv) Zₓ is as defined in paragraph (3) above, and/or
   R₂ₓ and R₃ₓ are as defined in paragraphs (8) and (9) respectively.

Suitably, in any of the compound groups 1 to 14 above, either:
Zₓ is as defined in paragraph (1) above, and/or
R₂ₓ and R₃ₓ are as defined in paragraphs (7) and (9) respectively.

Suitably, in any of the compound groups 1 to 14 above, R₁ₓ is as defined in paragraph (12) above.

Particular compounds of formula X that are of interest include any of the following, or a pharmaceutically acceptable salt thereof:
(1) Zₓ-[L-Lys]-[L-Dab(Z)]-[L-IgI]-[L-Arg]-R₁ₓ
(2) Zₓ-[L-Lys]-[L-Agp]-[L-IgI]-[L-Arg]-R₁ₓ
(3) Zₓ-[L-Lys]-[L-Glu(Bzl)]-[L-IgI]-[L-Arg]-R₁ₓ
(4) Zₓ-[L-Lys]-[L-Dab(Z)]-[L-Val]-[L-Arg]-R₁ₓ
(5) Zₓ-[L-Lys]-[L-Agp)]-[L-Val]-[L-Arg]-R₁ₓ
(6) Zₓ-[L-Lys]-[L-Glu(Bzl)]-[L-Val]-[L-Arg]-R₁ₓ
(7) Zₓ-[L-Lys]-[L-Dab(Z)]-[L-Lys]-[L-Arg]-R₁ₓ
(8) Zₓ-[L-Lys]-[L-Glu(Bzl)]-[L-Lys]-[L-Arg]-R₁ₓ
(9) Zₓ-[L-Lys]-[L-Glu(All)]-[L-IgI]-[L-Arg]-R₁ₓ;
wherein Zₓ and R₁ₓ each have any one of the definitions set out herein.

Specific compounds of formula X that are of interest include any of the following, or a pharmaceutically acceptable salt thereof:
(1) Ac-[L-Lys]-[L-Dab(Z)]-[L-IgI]-[L-Arg]-ACC (SMA5);
(2) Ac-[L-Lys]-[L-Agp]-[L-IgI]-[L-Arg]-ACC (SMA6);
(3) Ac-[L-Lys]-[L-Glu(Bzl)]-[L-IgI]-[L-Arg]-ACC (SMA7);
(4) Ac-[L-Lys]-[L-Dab(Z)]-[L-Val]-[L-Arg]-ACC (SMA9);
(5) Ac-[L-Lys]-[L-Agp]-[L-Val]-[L-Arg]-ACC (SMA10);
(6) Ac-[L-Lys]-[L-Glu(Bzl)]-[L-Val]-[L-Arg]-ACC (SMA11);
(7) Ac-[L-Lys]-[L-Dab(Z)]-[L-Lys]-[L-Arg]-ACC (SMA17);
(8) Ac-[L-Lys]-[L-Glu(Bzl)]-[L-Lys]-[L-Arg]-ACC (SMA19);
(9) Ac-[L-Lys]-[L-Glu(All)]-[L-IgI]-[L-Arg]-ACC (SMA25);
wherein Ac is acetyl and ACC is 7-amino-4-carbamoylmethylcoumarin.

Particular compounds of formula X that are of interest include any of the following, or a pharmaceutically acceptable salt thereof:
(1) Ac-[L-Lys]-[L-Dab(Z)]-[L-IgI]-[L-Arg]-ACC (SMA5);
(2) Ac-[L-Lys]-[L-Agp]-[L-IgI]-[L-Arg]-ACC (SMA6);
(3) Ac-[L-Lys]-[L-Dab(Z)]-[L-Lys]-[L-Arg]-ACC (SMA17);
wherein Ac is acetyl and ACC is 7-amino-4-carbamoylmethylcoumarin.

A particular compound of interest is:

Ac-[L-Lys]-[L-Dab(Z)]-[L-IgI]-[L-Arg]-ACC (SMA5);
wherein Ac is acetyl and ACC is 7-amino-4-carbamoylmethylcoumarin.

Though the present invention may relate to any compound or particular group of compounds defined herein by way of optional, preferred or suitable features, or otherwise in terms of particular embodiments, the present invention may also relate to any compound or particular group of compounds that specifically excludes said optional, preferred or suitable features or particular embodiments.

Suitably, the present invention excludes any individual compounds not possessing the biological activity defined herein.

Glossary of non-natural amino acids

| Amino Acid Name | Abbreviation | Structure |
|---|---|---|
| L-norleucine | L-NIe | 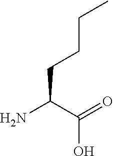 |
| L-homoarginine | L-hArg | 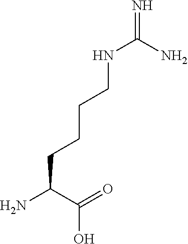 [SMILES: N=C(N)NCCCC[C@H](N)C(=O)O] |
| 4-amino-L-phenylalanine | L-Phe(4-NH₂) | 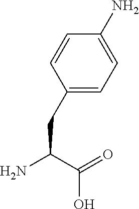 [SMILES: Nc1ccc(C[C@H](N)C(=O)O)cc1] |
| L-homoalanine | L-Abu | 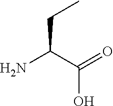 |
| L-phenylglycine | L-Phg | 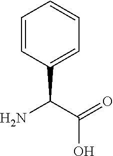 [SMILES: N[C@H](C(=O)O)c1ccccc1] |
| 2-indanyl-L-glycine | L-IgI | 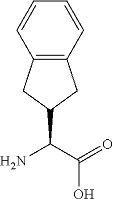 [SMILES: N[C@H](C(=O)O)C1Cc2ccccc2C1] |
| L-neopentyl-glycine | L-NptGly | 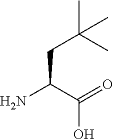 |
| L-norvaline | L-Nva | 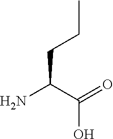 |
| benzyloxycarbonyl-L-2,4- diaminobutyric acid | L-Dab(Z) | 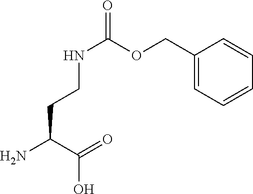 [SMILES: N[C@@H](CCNC(=O)OCc1ccccc1)C(=O)O] |
| D-homophenylalanine | D-hPhe | 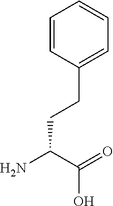 [SMILES: N[C@H](CCc1ccccc1)C(=O)O] |
| guanidino-L-alanine | L-Agp | 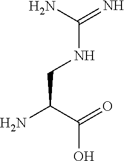 |
| L-glutamic acid benzyl ester | LGlu(BzI) | 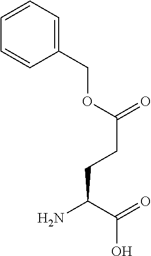 [SMILES: N[C@@H](CCC(=O)OCc1ccccc1)C(=O)O] |
| L-glutamic acid allyl ester | LGlu(All) | 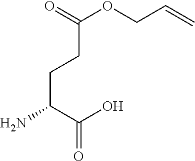 |
| L-methionine sulfone | L-Met(O)₂ | 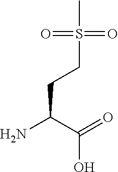 |
| L-biphenylalanine | L-Bip | 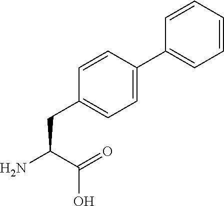 [SMILES: N[C@@H](Cc1ccc(-c2ccccc2)cc1)C(=O)O] |
| amino-L-homoalanine | L-Dab | 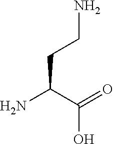 |
| N-trifluoroacetyl-L-lysine | L-Lys(TFA) | 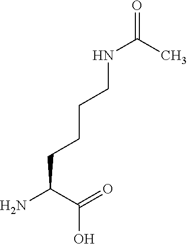 |
| N-2- chlorobenzyloxycarbonyl- L-lysine | L-Lys(2-Cl-Z) | 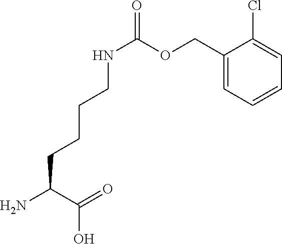 [SMILES: N[C@@H](CCCCNC(=O)OCc1ccccc1Cl)C(=O)O] |
| 4-iodo-L-phenylalanine | L-Phe(4-I) | 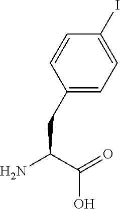 [SMILES: N[C@@H](Cc1ccc(I)cc1)C(=O)O] |
| 3-benzothienyl-L-alanine | L-Bta | 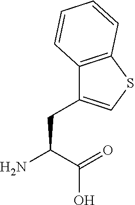 [SMILES: N[C@@H](Cc1csc2ccccc12)C(=O)O] |

### Salts and Solvates

The compounds (including final products and intermediates) described herein may be isolated and used per se or may be isolated in the form of a salt, suitably pharmaceutically acceptable salts. It should be understood that the terms “salt(s)” and “salt form(s)” used by themselves or in conjunction with another term or terms encompasses all inorganic and organic salts, including industrially acceptable salts, as defined herein, and pharmaceutically acceptable salts, as defined herein, unless otherwise specified. As used herein, industrially acceptable salts are salts that are generally suitable for manufacturing and/or processing (including purification) as well as for shipping and storage, but may not be salts that are typically administered for clinical or therapeutic use. Industrially acceptable salts may be prepared on a laboratory scale, i.e. multi-gram or smaller, or on a larger scale, i.e. up to and including a kilogram or more.

Pharmaceutically acceptable salts, as used herein, are salts that are generally chemically and/or physically compatible with the other ingredients comprising a formulation, and/or are generally physiologically compatible with the recipient thereof. Pharmaceutically acceptable salts may be prepared on a laboratory scale, i.e. multi-gram or smaller, or on a larger scale, i.e. up to and including a kilogram or more. It should be understood that pharmaceutically acceptable salts are not limited to salts that are typically administered or approved by the FDA or equivalent foreign regulatory body for clinical or therapeutic use in humans. A practitioner of ordinary skill will readily appreciate that some salts are both industrially acceptable as well as pharmaceutically acceptable salts. It should be understood that all such salts, including mixed salt forms, are within the scope of the application.

In one embodiment, the compounds of Formula I or X and sub-formulae thereof are isolated as pharmaceutically acceptable salts.

A suitable pharmaceutically acceptable salt of a compound of the invention is, for example, an acid-addition salt of a compound of the invention which is sufficiently basic, for example, an acid-addition salt with, for example, an inorganic or organic acid, for example hydrochloric, hydrobromic, sulfuric, phosphoric, trifluoroacetic, formic, citric or maleic acid. In addition a suitable pharmaceutically acceptable salt of a compound of the invention which is sufficiently acidic is an alkali metal salt, for example a sodium or potassium salt, an alkaline earth metal salt, for example a calcium or magnesium salt, an ammonium salt or a salt with an organic base which affords a physiologically-acceptable cation, for example a salt with methylamine, dimethylamine, trimethylamine, piperidine, morpholine or tris-(2-hydroxyethyl)amine.

In general, salts of the present application can be prepared in situ during the isolation and/or purification of a compound (including intermediates), or by separately reacting the compound (or intermediate) with a suitable organic or inorganic acid or base (as appropriate) and isolating the salt thus formed. The degree of ionisation in the salt may vary from completely ionised to almost non-ionised. In practice, the various salts may be precipitated (with or without the addition of one or more co-solvents and/or anti-solvents) and collected by filtration or the salts may be recovered by evaporation of solvent(s). Salts of the present application may also be formed via a “salt switch” or ion exchange/double displacement reaction, i.e. reaction in which one ion is replaced (wholly or in part) with another ion having the same charge. One skilled in the art will appreciate that the salts may be prepared and/or isolated using a single method or a combination of methods.

Representative salts include, but are not limited to, acetate, aspartate, benzoate, besylate, bicarbonate/carbonate, bisulphate/sulphate, borate, camsylate, citrate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, palmitate, oxalate, pamoate, phosphate/hydrogen phosphate/dihydrogen phosphate, saccharate, stearate, succinate, tartrate, tosylate, trifluoroacetate and the like. Other examples of representative salts include alkali or alkaline earth metal cations such as, but not limited to, sodium, lithium, potassium, calcium, magnesium, and the like, as well as non-toxic ammonium, quaternary ammonium and amine cations including, but not limited to, ammonium, tetramethylammonium, tetraethylammonium, lysine, arginine, benzathine, choline, tromethamine, diolamine, glycine, meglumine, olamine and the like.

Certain compounds of the Formula I or X and sub-formulae thereof may exist in solvated as well as unsolvated forms such as, for example, hydrated forms. It is to be understood that the invention encompasses all such solvated forms that possess the biological activity described herein.

### Polymorphs

It is also to be understood that certain compounds of the Formula I or X and sub-formulae thereof may exhibit polymorphism, and that the invention encompasses all such forms that possess the biological activity described herein.

### Isomers

Compounds that have the same molecular formula but differ in the nature or sequence of bonding of their atoms or the arrangement of their atoms in space are termed “isomers”. Isomers that differ in the arrangement of their atoms in space are termed “stereoisomers”. Stereoisomers that are not mirror images of one another are termed “diastereomers” and those that are non-superimposable mirror images of each other are termed “enantiomers”. When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

Certain compounds of Formula I or X and sub-formulae thereof may have one or more asymmetric centres and therefore can exist in a number of stereoisomeric configurations. Consequently, such compounds can be synthesized and/or isolated as mixtures of enantiomers and/or as individual (pure) enantiomers, and, in the case of two or more asymmetric centres, single diastereomers and/or mixtures of diastereomers. It should be understood that the present application includes all such enantiomers and diastereomers and mixtures thereof in all ratios.

### Isotopes

The compounds of the present invention are described herein using structural formulas that do not specifically recite the mass numbers or the isotope ratios of the constituent atoms. As such it is intended that the present application includes compounds in which the constituent atoms are present in any ratio of isotope forms. For example, carbon atoms may be present in any ratio of ¹²C, ¹³C, and ¹⁴C; hydrogen atoms may be present in any ratio of ¹H, ²H, and ³H; etc. Preferably, the constituent atoms in the compounds of the present invention are present in their naturally occurring ratios of isotope forms.

### Prodrugs and Metabolites

The compounds of Formula I and X may be administered in the form of a pro-drug which is broken down in the human or animal body to release a compound of the invention. A pro-drug may be used to alter the physical properties and/or the pharmacokinetic properties of a compound of the invention. A pro-drug can be formed when the compound of the invention contains a suitable group or substituent to which a property-modifying group can be attached. Examples of pro-drugs include in vivo cleavable ester derivatives that may be formed at a carboxy group or a hydroxy group in a compound of the Formula I and X and in-vivo cleavable amide derivatives that may be formed at a carboxy group or an amino group in a compound of the Formula I and X. Compounds of the invention comprising an arginine residue may also comprise a N-omega-hydroxy prodrug group, as shown below:

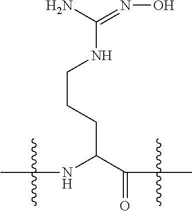 [SMILES: *NC(CCCN/C(N)=N\O)C(*)=O]

The N-omega-hydroxy prodrug group is cleaved in vivo to form arginine.

Accordingly, the present invention includes those compounds of the Formula I and X as defined hereinbefore when made available by organic synthesis and when made available within the human or animal body by way of cleavage of a pro-drug thereof. Accordingly, the present invention includes those compounds of the Formula I and X that are produced by organic synthetic means and also such compounds that are produced in the human or animal body by way of metabolism of a precursor compound, that is a compound of the Formula I and X may be a synthetically-produced compound or a metabolically-produced compound.

A suitable pharmaceutically acceptable pro-drug of a compound of the Formula I and X is one that is based on reasonable medical judgement as being suitable for administration to the human or animal body without undesirable pharmacological activities and without undue toxicity.

Various forms of pro-drug have been described, for example in the following documents:—
a) Methods in Enzymology, Vol. 42, p. 309-396, edited by K. Widder, et al. (Academic Press, 1985);
b) Design of Pro-drugs, edited by H. Bundgaard, (Elsevier, 1985);
c) A Textbook of Drug Design and Development, edited by Krogsgaard-Larsen and H. Bundgaard, Chapter 5 “Design and Application of Pro-drugs”, by H. Bundgaard p. 113-191 (1991);
d) H. Bundgaard, Advanced Drug Delivery Reviews, 8, 1-38 (1992);
e) H. Bundgaard, et al., Journal of Pharmaceutical Sciences, 77, 285 (1988);
f) N. Kakeya, et al., Chem. Pharm. Bull., 32, 692 (1984);
g) T. Higuchi and V. Stella, “Pro-Drugs as Novel Delivery Systems”, A.C.S. Symposium Series, Volume 14; and
h) E. Roche (editor), “Bioreversible Carriers in Drug Design”, Pergamon Press, 1987.

A suitable pharmaceutically acceptable pro-drug of a compound of the Formula I and X that possesses a carboxy group is, for example, an in vivo cleavable ester thereof. An in vivo cleavable ester of a compound of the Formula I and X containing a carboxy group is, for example, a pharmaceutically acceptable ester which is cleaved in the human or animal body to produce the parent acid. Suitable pharmaceutically acceptable esters for carboxy include C₁₋₆alkyl esters such as, but not limited to, methyl, ethyl and tert-butyl, C₁₋₆alkoxymethyl esters such as, but not limited to, methoxymethyl esters, C₁₋₆alkanoyloxymethyl esters such as, but not limited to, pivaloyloxymethyl esters, 3-phthalidyl esters, C₃₋₈cycloalkylcarbonyloxy-C₁₋₆alkyl esters such as, but not limited to, cyclopentylcarbonyloxymethyl and 1-cyclohexylcarbonyloxyethyl esters, 2-oxo-1,3-dioxolenylmethyl esters such as, but not limited to, 5-methyl-2-oxo-1,3-dioxolen-4-ylmethyl esters and C₁₋₆alkoxycarbonyloxy-C₁₋₆alkyl esters such as, but not limited to, methoxycarbonyloxymethyl and 1-methoxycarbonyloxyethyl esters.

A suitable pharmaceutically acceptable pro-drug of a compound of the Formula I and X that possesses a hydroxy group is, for example, an in vivo cleavable ester or ether thereof. An in vivo cleavable ester or ether of a compound of the Formula I and X containing a hydroxy group is, for example, a pharmaceutically acceptable ester or ether which is cleaved in the human or animal body to produce the parent hydroxy compound. Suitable pharmaceutically acceptable ester forming groups for a hydroxy group include inorganic esters such as, but not limited to, phosphate esters (including phosphoramidic cyclic esters). Further suitable pharmaceutically acceptable ester forming groups for a hydroxy group include C₁₋₁₀alkanoyl groups such as, but not limited to, acetyl, benzoyl, phenylacetyl and substituted benzoyl and phenylacetyl groups, C₁₋₁₀alkoxycarbonyl groups such as, but not limited to, ethoxycarbonyl, N,N—(C₁₋₆)₂carbamoyl, 2-dialkylaminoacetyl and 2-carboxyacetyl groups. Examples of ring substituents on the phenylacetyl and benzoyl groups include aminomethyl, N-alkylaminomethyl, N,N-dialkylaminomethyl, morpholinomethyl, piperazin-1-ylmethyl and 4-(C₁₋₄alkyl) piperazin-1-ylmethyl. Suitable pharmaceutically acceptable ether forming groups for a hydroxy group include α-acyloxyalkyl groups such as, but not limited to, acetoxymethyl and pivaloyloxymethyl groups.

A suitable pharmaceutically acceptable pro-drug of a compound of the Formula I and X that possesses a carboxy group is, for example, an in vivo cleavable amide thereof, for example an amide formed with an amine such as, but not limited to, ammonia, a C₁₋₄alkylamine such as, but not limited to, methylamine, a (C₁₋₄alkyl)₂amine such as, but not limited to, dimethylamine, N-ethyl-N-methylamine or diethylamine, a C₁₋₄alkoxy-C₂₋₄alkylamine such as, but not limited to, 2-methoxyethylamine, a phenyl-C₁₋₄alkylamine such as, but not limited to, benzylamine and amino acids such as, but not limited to, glycine or an ester thereof.

A suitable pharmaceutically acceptable pro-drug of a compound of the Formula I and X that possesses an amino group is, for example, an in vivo cleavable amide derivative thereof. Suitable pharmaceutically acceptable amides from an amino group include, for example an amide formed with C₁₋₁₀alkanoyl groups such as, but not limited to, an acetyl, benzoyl, phenylacetyl and substituted benzoyl and phenylacetyl groups. Examples of ring substituents on the phenylacetyl and benzoyl groups include aminomethyl, N-alkylaminomethyl, N,N-dialkylaminomethyl, morpholinomethyl, piperazin-1-ylmethyl and 4-(C₁₋₄alkyl) piperazin-1-ylmethyl.

The in vivo effects of a compound of the Formula I and X may be exerted in part by one or more metabolites that are formed within the human or animal body after administration of a compound of the Formula I and X. As stated hereinbefore, the in vivo effects of a compound of the Formula I and X may also be exerted by way of metabolism of a precursor compound (a pro-drug).

### Compositions

According to a further aspect of the invention there is provided a composition (e.g. a pharmaceutical composition) which comprises a compound of the invention as defined hereinbefore, or a salt, hydrate or solvate thereof, in association with a diluent or carrier.

The pharmaceutical compositions of the invention may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous, intramuscular, intraperitoneal or intramuscular dosing or as a suppository for rectal dosing).

The compositions of the invention may be obtained by conventional procedures using conventional excipients, well known in the art. Thus, compositions intended for oral use may contain, for example, one or more colouring, sweetening, flavouring and/or preservative agents.

An effective amount of a compound of the present invention for use in therapy is an amount sufficient to treat or prevent a condition referred to herein, slow its progression and/or reduce the symptoms associated with the condition.

The amount of active ingredient that is combined with one or more excipients to produce a single dosage form will necessarily vary depending upon the individual treated and the particular route of administration. For example, a formulation intended for oral administration to humans will generally contain, for example, from 0.5 mg to 1.5 g of active agent (more suitably from 0.5 to 600 mg, for example from 1 to 200 mg) compounded with an appropriate and convenient amount of excipients which may vary from about 5 to about 98 percent by weight of the total composition.

The size of the dose for therapeutic or prophylactic purposes of a compound of the Formula I or X will naturally vary according to the nature and severity of the conditions, the age and sex of the animal or patient and the route of administration, according to well-known principles of medicine.

It is to be noted that dosages and dosing regimens may vary with the type and severity of the condition to be alleviated, and may include the administration of single or multiple doses, i.e. QD (once daily), BID (twice daily), etc., over a particular period of time (days or hours). It is to be further understood that for any particular subject or patient, specific dosage regimens may need to be adjusted over time according to the individual need and the professional judgment of the person administering or supervising the administration of the pharmaceutical compositions. For example, doses may be adjusted based on pharmacokinetic or pharmacodynamic parameters, which may include clinical effects such as toxic effects and/or laboratory values. Thus, the present application encompasses intra-patient dose-escalation as determined by the person skilled in the art. Procedures and processes for determining the appropriate dosage(s) and dosing regimen(s) are well-known in the relevant art and would readily be ascertained by the skilled artisan. As such, one of ordinary skill would readily appreciate and recognize that the dosage ranges set forth herein are exemplary only and are not intended to limit the scope or practice of the pharmaceutical compositions described herein.

In using a compound of the invention for therapeutic or prophylactic purposes it will generally be administered so that a daily dose in the range, for example, 0.1 mg/kg to 75 mg/kg body weight is received, given if required in divided doses. In general lower doses will be administered when a parenteral route is employed. Thus, for example, for intravenous or intraperitoneal administration, a dose in the range, for example, 0.1 mg/kg to 30 mg/kg body weight will generally be used. Similarly, for administration by inhalation, a dose in the range, for example, 0.05 mg/kg to 25 mg/kg body weight will be used.

### Synthesis

The compounds of the present invention can be prepared by any suitable technique known in the art. Particular methods for forming compounds of Formula I or X defined herein are described or referenced in the accompanying example section.

In the description of the synthetic methods described herein and in any referenced synthetic methods that are used to prepare the starting materials, it is to be understood that all proposed reaction conditions, including choice of solvent, reaction atmosphere, reaction temperature, coupling reagents, duration of the experiment and workup procedures, can be selected by a person skilled in the art.

It is understood by one skilled in the art of organic synthesis that the functionality present on various portions of the molecule must be compatible with the reagents and reaction conditions utilised.

It will be appreciated that during the synthesis of the compounds of the invention in the processes defined herein, or during the synthesis of certain starting materials, it may be desirable to protect certain substituent groups to prevent their undesired reaction. The skilled chemist will appreciate when such protection is required, and how such protecting groups may be put in place, and later removed.

For Examples of protecting groups see one of the many general texts on the subject, for example, ‘Protective Groups in Organic Synthesis’ by Theodora Green (publisher: John Wiley & Sons). Protecting groups may be removed by any convenient method described in the literature or known to the skilled chemist as appropriate for the removal of the protecting group in question, such methods being chosen so as to effect removal of the protecting group with the minimum disturbance of groups elsewhere in the molecule.

Thus, if reactants include, for example, groups such as amino, carboxy or hydroxy it may be desirable to protect the group in some of the reactions mentioned herein.

By way of example, a suitable protecting group for an amino or alkylamino group is, for example, an acyl group, for example an alkanoyl group such as, but not limited to, acetyl, an alkoxycarbonyl group, for example a methoxycarbonyl, ethoxycarbonyl or tertbutoxycarbonyl group, an arylmethoxycarbonyl group, for example benzyloxycarbonyl, or an aroyl group, for example benzoyl. The deprotection conditions for the above protecting groups necessarily vary with the choice of protecting group. Thus, for example, an acyl group such as an alkanoyl or alkoxycarbonyl group or an aroyl group may be removed by, for example, hydrolysis with a suitable base such as, but not limited to, an alkali metal hydroxide, for example lithium or sodium hydroxide. Alternatively an acyl group such as a tertbutoxycarbonyl group may be removed, for example, by treatment with a suitable acid as hydrochloric, sulfuric or phosphoric acid or trifluoroacetic acid and an arylmethoxycarbonyl group such as a benzyloxycarbonyl group may be removed, for example, by hydrogenation over a catalyst such as palladium on carbon, or by treatment with a Lewis acid for example boron tris(trifluoroacetate). A suitable alternative protecting group for a primary amino group is, for example, a phthaloyl group which may be removed by treatment with an alkylamine, for example dimethylaminopropylamine, or with hydrazine.

A suitable protecting group for a hydroxy group is, for example, an acyl group, for example an alkanoyl group such as acetyl, an aroyl group, for example benzoyl, or an arylmethyl group, for example benzyl. The deprotection conditions for the above protecting groups will necessarily vary with the choice of protecting group. Thus, for example, an acyl group such as an alkanoyl or an aroyl group may be removed, for example, by hydrolysis with a suitable base such as an alkali metal hydroxide, for example lithium, sodium hydroxide or ammonia. Alternatively, an arylmethyl group such as a benzyl group may be removed, for example, by hydrogenation over a catalyst such as palladium on carbon.

A suitable protecting group for a carboxy group is, for example, an esterifying group, for example a methyl or an ethyl group which may be removed, for example, by hydrolysis with a base such as sodium hydroxide, or for example a tertbutyl group which may be removed, for example, by treatment with an acid, for example an organic acid such as trifluoroacetic acid, or for example a benzyl group which may be removed, for example, by hydrogenation over a catalyst such as palladium on carbon.

Resins may also be used as a protecting group.

### Uses and Applications

A further aspect of the invention provides the use of a compound according to the invention, as defined above, in the detection or inhibition ex-vivo of a blood coagulation protease, wherein said compound is an inhibitor of one or more coagulation proteases selected from APC, fIIa, fXa and fXIa.

A further aspect of the invention provides a compound of the invention, as defined above, for use in the inhibition of blood coagulation wherein said compound is acting as inhibitor of one or more coagulation proteases selected from fIIa, fXa and fXIa.

A further aspect of the invention provides a compound of the invention, as defined above, for use in treatment or prevention of the illness connected with coagulation protease activity, wherein said compound is acting as inhibitor of one or more coagulation proteases selected from APC, fIIa, fXa and fXIa.

Preferably, said illness has been selected from the group consisting of: bleeding disorders (e.g. hemophilia), thrombosis, disseminated intravascular coagulation, ischemic stroke, myocardial infarction, cancer, Alzheimer's disease, sepsis, multiple sclerosis and COVID-19.

Preferably, said compound is acting as inhibitor of APC and is represented by the chemical formula 2 as defined above.

Preferably, said compound is acting as inhibitor of fIIa and is represented by the chemical formula 3 as defined above.

Preferably, said compound is acting as inhibitor of fXa and is represented by the chemical formula 4 as defined above.

Preferably, said compound is acting as inhibitor of fXIa and is represented by the chemical formula 5 as defined above.

The disclosed inhibitors can be applied for the pharmacological knockdown of individual enzymes and fluorescent ABPs can be used to monitor and detect the level of APC, fIIa, fXa, and fXIa in biological samples. Since coagulation proteases serve as markers for multiple disorders, ABPs according to the invention may be used as diagnostic tools and facilitate the choice of appropriate therapy for diseases like hemophilia, thrombosis, stroke, sepsis, and many others.

In a particular aspect of the invention, the compound of Formula X according to the third aspect of the invention, and said compound functions as an inhibitor of APC.

### Numbered Clauses:

Clause 1. A compound represented by the following chemical formula 1:

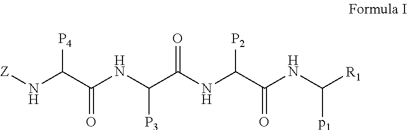

wherein:
   Z is acetyl (Ac) or group of formula R₃-R₂-
      wherein: R₂ is linker group such as: PEG or 6-aminohexanoic acid; and
      R₃ is fluorescent group such as: 1-(5-carboxypentyl)-3,3-dimethyl-2-((1E,3Z)-3-(1,3,3-trimethylindolin-2-ylidene) prop-1-enyl)-3H-indolium (Cyanine 3), 1-(5-carboxypentyl)-2-((1E,3E,5E)-5-(1,3-dimethylindolin-2-ylidene) penta-1,3-dienyl)-3,3-dimethyl-3H-indolium (Cyanine 5), 1-(5-carboxypentyl)-3,3-dimethyl-2-((E)-2-((E)-3-((Z)-2-(1,3,3-trimethylindolin-2-ylidene)ethylidene)cyclohex-1-enyl) vinyl)-3H-indolium (Cyanine 7), or 4,4-difluoro-5,7-dimethyl-4-bora-3a,4a-diaza-s-indacene-3-propionic acid (Bodipy)
   R₁ is reactive binding group such as: diphenyl phosphonate and its derivatives
   P₁ is functional group:

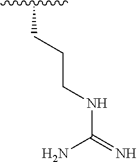

L-Arginine (L-Arg) side chain;
P₂ is a functional group selected among of:

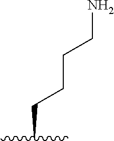

L-Lysine (L-Lys) side chain,

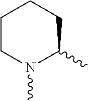

L-piperidine-2-carboxylic acid (L-Pip) side chain,

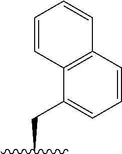

3-(1-naphthyl)-L-alanine (L-1-Nal) side chain,

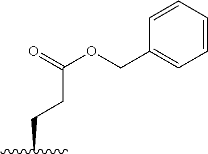

L-glutamic-acid-gamma-benzyl ester (L-Glu(Bzl)) side chain,
P₃ is functional group selected among of:

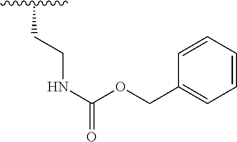

benzyloxycarbonyl-L-2,4-diaminobutyric acid (L-Dab(Z)) side chain,

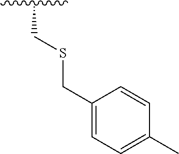

4-methylbenzyl-L-cysteine (L-Cys(MeBzl)) side chain,

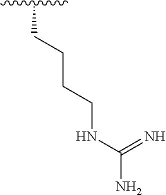

L-homoarginine (L-hArg) side chain,

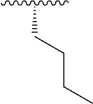

L-norleucine (L-Nle) side chain,
P₄ is functional group selected among of:

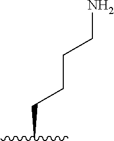

L-Lysine (L-Lys) side chain,

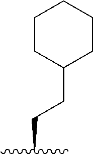

L-homocyclohexylalanine (L-hCha) side chain,

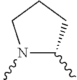

D-proline (D-Pro) side chain,

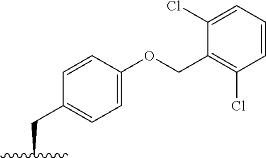

2,6-dichlorobenzyl-L-tyrosine (L-Tyr (2,6-Cl₂—Z) side chain.
Clause 2. A compound according to clause 1, characterized in that it has been selected among of compounds represented by the following chemical formulas 2-5:

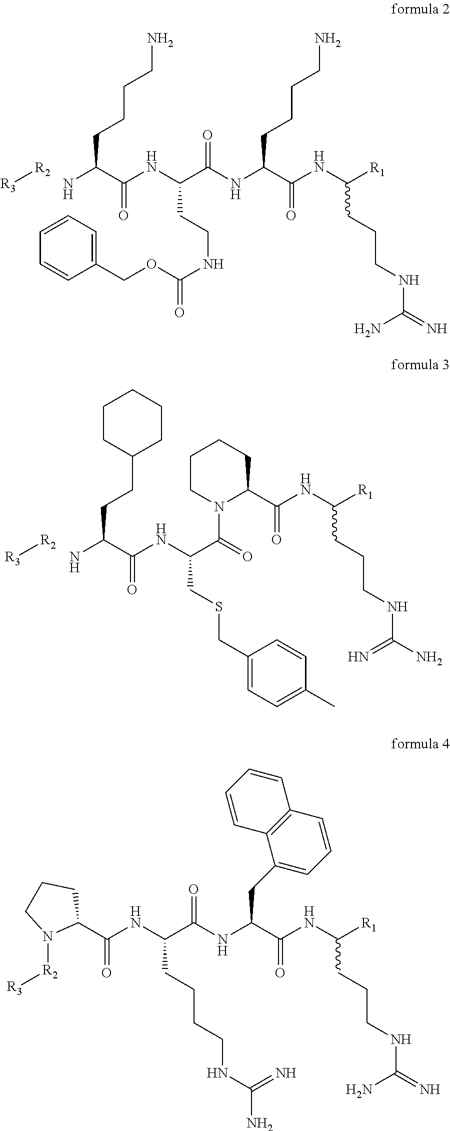

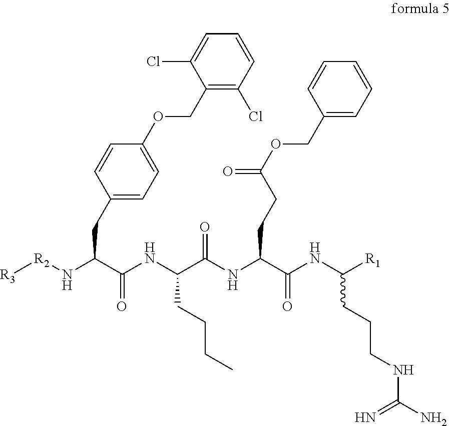

wherein:
R₃ is acetyl (Ac) or fluorescent group such as: 1-(5-carboxypentyl)-3,3-dimethyl-2-((1E,3Z)-3-(1,3,3-trimethylindolin-2-ylidene) prop-1-enyl)-3H-indolium (Cyanine 3), 1-(5-carboxypentyl)-2-((1E,3E,5E)-5-(1,3-dimethylindolin-2-ylidene) penta-1,3-dienyl)-3,3-dimethyl-3H-indolium (Cyanine 5), 1-(5-carboxypentyl)-3,3-dimethyl-2-((E)-2-((E)-3-((Z)-2-(1,3,3-trimethylindolin-2-ylidene)ethylidene)cyclohex-1-enyl) vinyl)-3H-indolium (Cyanine 7), or 4,4-difluoro-5,7-dimethyl-4-bora-3a,4a-diaza-s-indacene-3-propionic acid (Bodipy), R₂ is linker group such as: PEG or 6-aminohexanoic acid, R₁ is reactive binding group such as: diphenyl phosphonate and its derivatives.
Clause 3: A compound according to clause 1 or 2, wherein it has been selected from the following compounds:
   Cy5-6-Ahx-Lys-Dab(Z)-Lys-Arg^{P}(OPh)₂,
   Ac-Lys-Dab(Z)-Lys-Arg^{P}(OPh)₂,
   Cy7-6-Ahx-hCha-Cys(MeBzl)-Pip-Arg^{P}(OPh)₂,
   Ac-hCha-Cys(MeBzl)-Pip-Arg^{P}(OPh)₂,
   Cy3-6-Ahx-DPro-hArg-1-Nal-Arg^{P}(OPh)₂,
   Ac-DPro-hArg-1-Nal-Arg^{P}(OPh)₂,
   Bodipy-PEG (4)-Tyr (2,6Cl₂—Z)-Nie-Glu(Bzl)-Arg^{P}(OPh)₂ and
   Ac-Tyr(2,6Cl₂—Z)-Nle-Glu(Bzl)-Arg^{P}(OPh)₂.
Clause 4: Use of a compound according to any preceding clause for the detection or inhibition ex-vivo of blood coagulation protease, wherein said compound is inhibitor of coagulation protease selected from: APC, fIIa, fXa and fXIa.
Clause 5: A compound according to any of clauses 1 to 3 for use in the inhibition of blood coagulation wherein said compound is acting as inhibitor of coagulation protease selected from: fIIa, fXa and fXIa.
Clause 6: A compound according to any of clauses 1 to 3 for use in treatment or prevention of an illness connected with, or associated with, abnormal coagulation protease activity, wherein said compound is acting as inhibitor of coagulation protease selected from: APC, fIIa, fXa and fXIa.
Clause 7: A compound for use according to clause 6 wherein said illness has been selected from the group consisting of: hemophilia, thrombosis, disseminated intravascular coagulation, ischemic stroke, myocardial infarction, cancer, Alzheimer's disease, sepsis, multiple sclerosis and COVID-19.
Clause 8: Use of a compound according to clause 4, or a compound for use according to clause 5 or 6, wherein said compound is acting as inhibitor of APC and is represented by the chemical formula 2 as defined in clause 2.
Clause 9: Use of a compound according to clause 4, or a compound for use according to clause 5 or 6 wherein said compound is acting as inhibitor of fIIa and is represented by the chemical formula 3 as defined in clause 2.
Clause 10: Use of a compound according to clause 4, or a compound for use according to clause 5 or 6, wherein said compound is acting as inhibitor of fXa and is represented by the chemical formula 4 as defined in clause 2.
Clause 11: Use of a compound according to clause 4, or a compound for use according to clause 5 or 6, wherein said compound is acting as an inhibitor of fXIa and is represented by the chemical formula 5 as defined in clause 2.

### EXAMPLES

### Example 1. Obtaining of Fluorescent Activity Based Probes (ABPs) and Inhibitors Activity-Based Probe and Inhibitor Synthesis

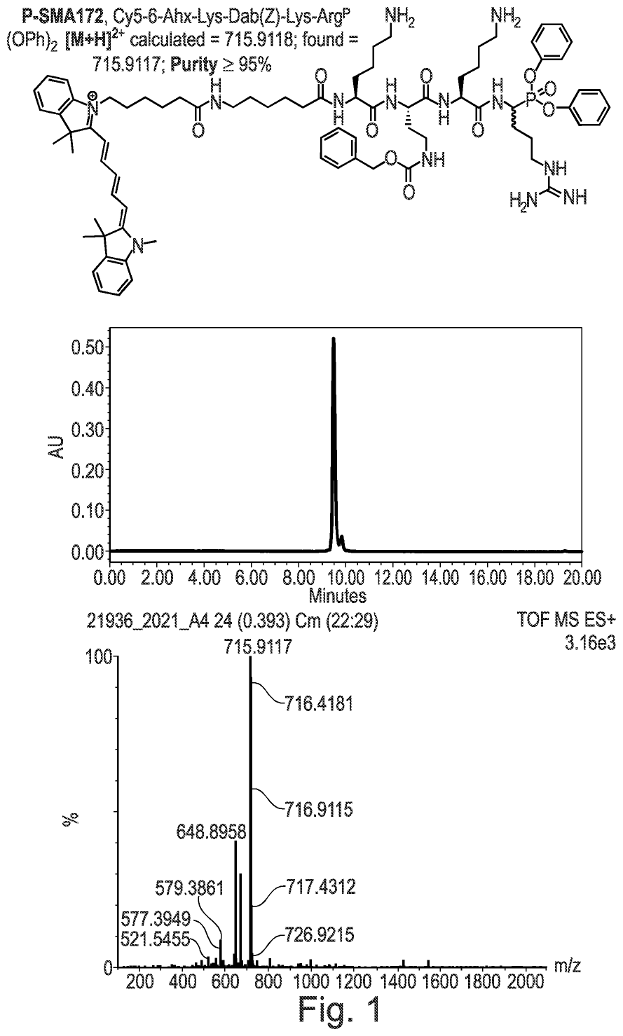

Designed chemical markers are equipped with the fluorophores to namely Cy3, Cy5, Cy7 or BodipyFL that are separated from the peptide sequence by 6-aminohexanoic acid or polyethylene glycol linker. As a reactive warhead a diphenyl phosphonate was selected. Inhibitors contain acetylated peptide sequences (the same sequence as in the fluorescent ABPs) and a diphenyl phosphonate as an irreversible reactive group. Fluorescent ABPs and inhibitors (FIG. 1) were synthesized using mixed solid- and solution-phase approach. In the first step 2-chlorotrityl resin was placed in the peptide synthesis vessel and swollen in anhydrous DCM for 30 min, and then, the resin was washed once with DCM. Next, Fmoc-P2-OH (2.5 equiv) was dissolved in in anhydrous DCM, preactivated with DIPEA (3 equiv), added to the resin under an argon atmosphere, and stirred gently for 12 h. After that, the mixture was filtered, the resin was washed with DCM (three times), and the remaining active sites on the 2-chlorotrityl resin were deactivated with DCM/MeOH/DIPEA (%, v/v/v, 80:15:5) solution, for 1 h. Next, the mixture was filtered, the resin was washed with DMF (six times), and N-terminus Fmoc-protecting group was removed using 20% piperidine in DMF (5, 5, and 25 min). Afterwards, the P3, P4 amino acids, and Fmoc-Ahx-OH/Fmoc-PEG (4)-OH were attached stepwise to the H₂N-P2-resin with HOBt (2.5 equiv), and DICl (2.5 equiv) as coupling reagents in coupling/deprotection cycles. In the case of fluorescently-labeled probes to the amine group of H₂N-Ahx/PEG (4)-P4-P3-P2-resin the fluorescent tag was coupled (0.3 equiv). Fluorophore was dissolved in DMF, preactivated with DIPEA (1.2 equiv), and stirred with the resin overnight. Next, the resin was washed with DMF (six times), DCM (three times), MeOH (three times), and dried over P₂O₅. The crude peptide was cleaved from the resin with a mixture of DCM/TFE/AcOH (%, v/v/v, 80:10:10). The solution was filtered, concentrated, and lyophilized. Next, diphenyl phosphonate (Cbz-Arg (Boc)₂^{P}(OPh)₂) was synthesized according to a previously described methodology [25]. The Cbz-protecting group was removed using hydrogen and palladium on carbon. The obtained H₂N-Arg (Boc) 2^{P}(OPh)₂ (1 equiv) was coupled with a peptide sequence (1.2 equiv), with HATU (1.2 equiv) and 2,4,6-collidine (4 equiv). The reaction was monitored with analytical HPLC and after 2 h the product was extracted to ethyl acetate with 5% NaHCO₃, 5% citric acid, and brine. The organic phase was collected, dried over MgSO₄, and evaporated. Finally, the side chain amino acid protecting groups were removed with a mixture of TFA/DCM/TIPS (%, v/v/v, 80:15:5). After 30 min solvents were removed with argon flow, and the obtained product was dissolved in peptide grade DMSO, purified on HPLC, and lyophilized. The ABP purity was confirmed by analytical HPLC, analyzed using HRMS. ABP was then dissolved in peptide grade DMSO to a final concentration of 10 mM and stored at −80° C.

To obtain inhibitors, the amino group of H₂N-P4-P3-P2-resin was acetylated using 5 equiv of AcOH, 5 equiv of HBTU, and 5 equiv of DIPEA in DMF. After 1 h the solution was filtered, the resin was washed with DMF (six times), DCM (three times), MeOH (three times), and dried over P₂O₅. The remaining steps were analogous to the synthesis of the fluorescent ABPs.

All individual ABPs and inhibitors were purified by HPLC (Waters M600 solvent delivery module, Waters M2489 detector system, semipreparative Wide Pore C8 Discovery column, Waters sp z.o.o., Warszawa, Poland). The solvent composition was as follows: phase A (water: 0.1% TFA) and phase B (acetonitrile: 0.1% TFA). The purity of each compound was confirmed with an analytical HPLC system using a Discovery Bio Wide Pore C8 analytical column. The solvent composition was as follows: water: 0.1% TFA for phase A and acetonitrile: 0.1% TFA for phase B; gradient, from 95% A to 5% A over a period of 15 min. The purity of all compounds was ≥95%. The molecular weight of each compound was confirmed by high-resolution mass spectrometry on a WATERS LCT premier XE with electrospray ionization (ESI) and a time-of-flight (TOF) module.

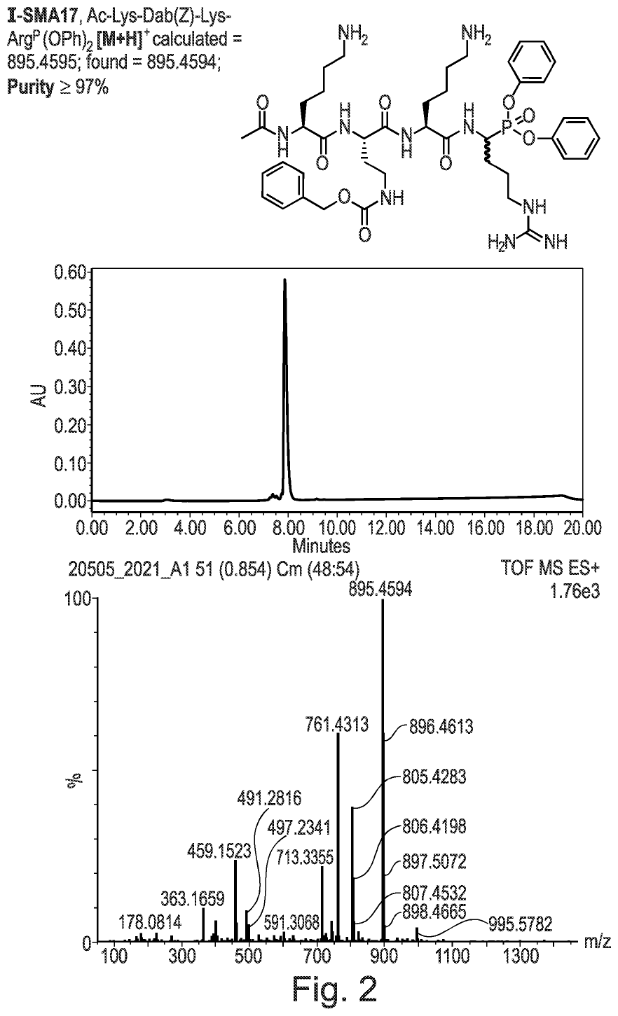

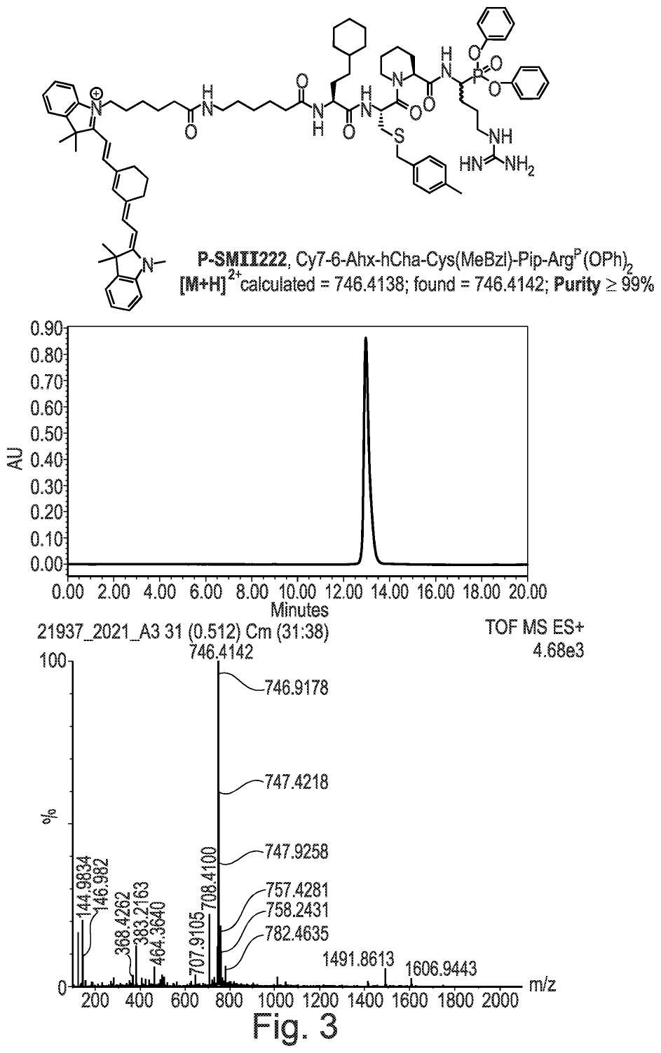

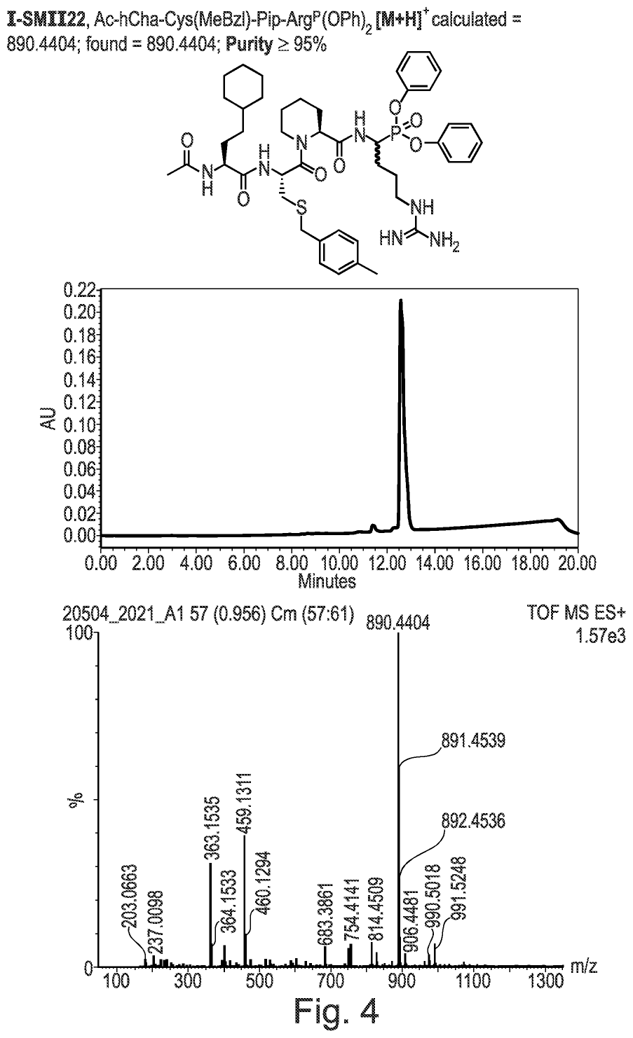

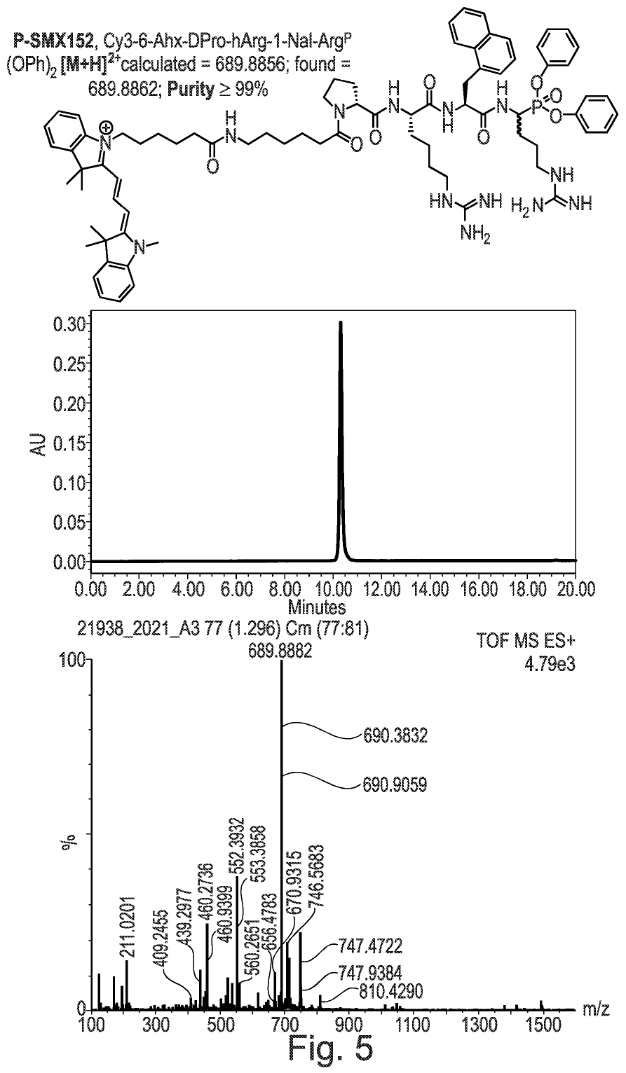

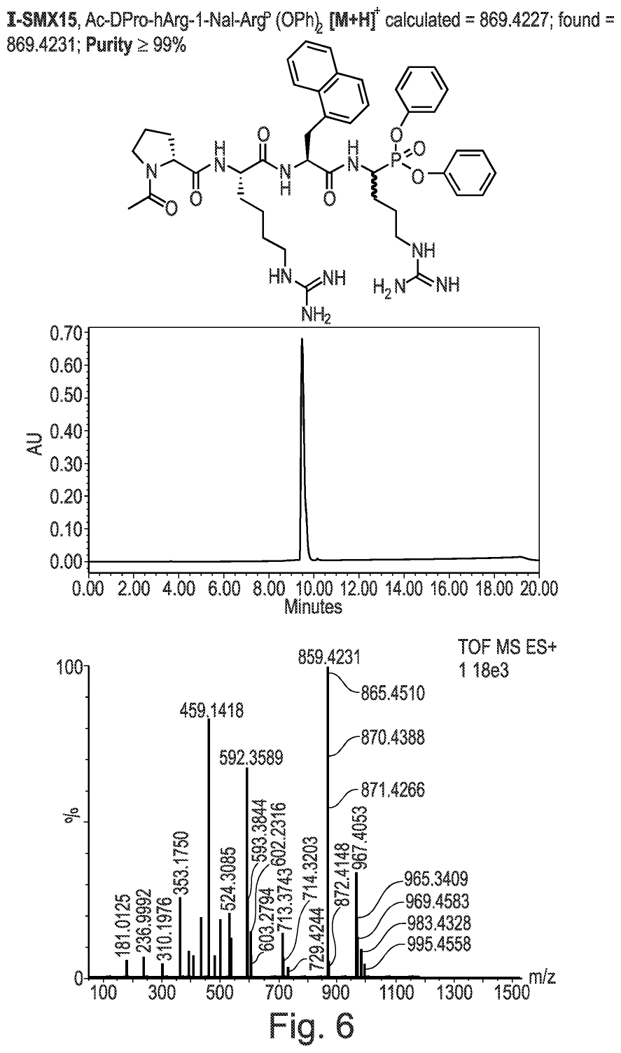

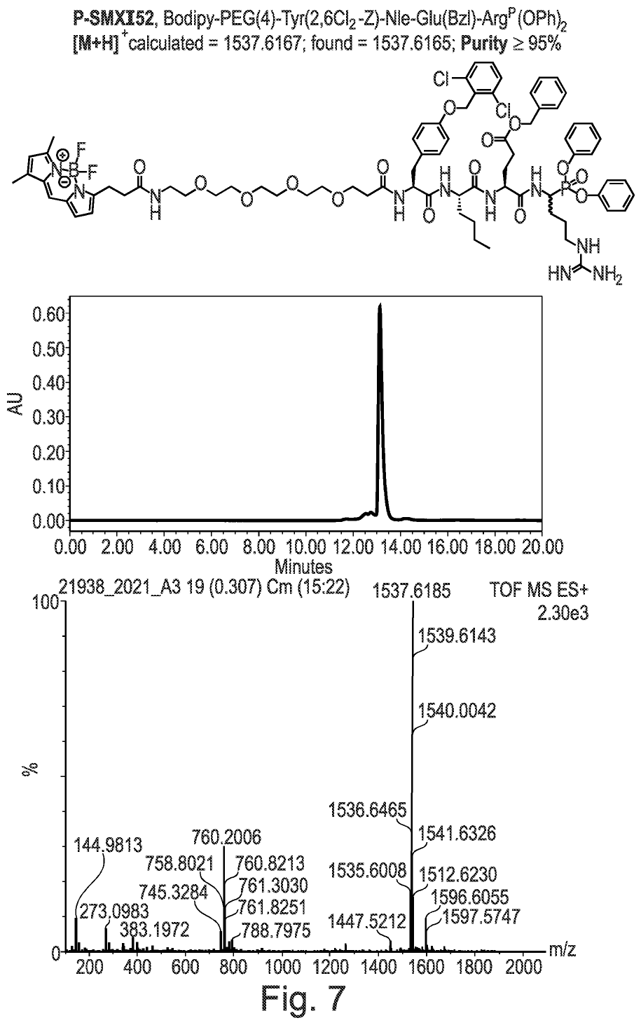

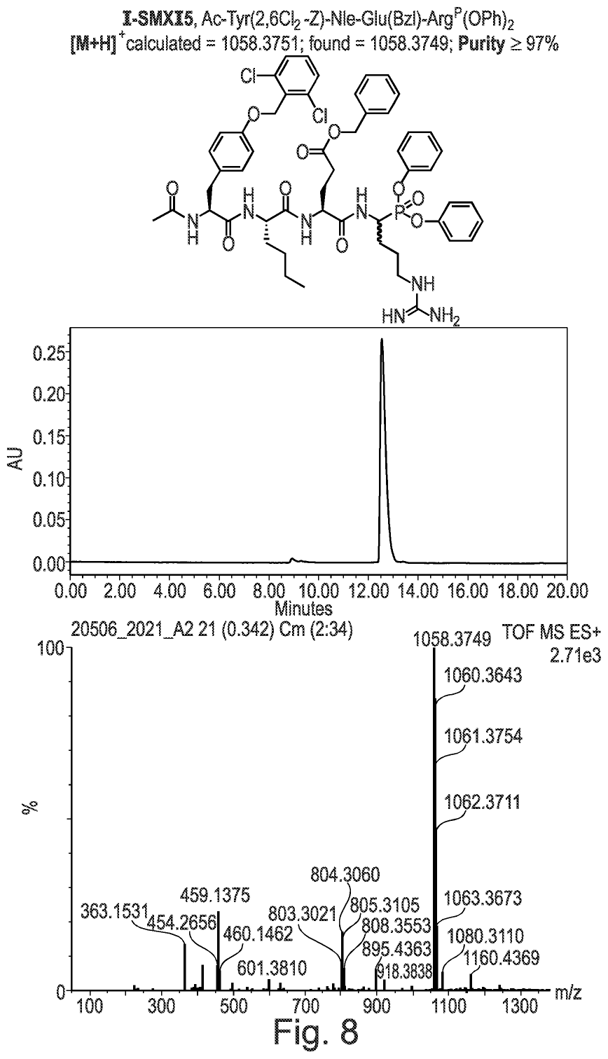

Results obtained for compounds: Cy5-6-Ahx-Lys-Dab(Z)-Lys-Arg^{P}(OPh)₂, Ac-Lys-Dab(Z)-Lys-Arg^{P}(OPh)₂, Cy7-6-Ahx-hCha-Cys(MeBzl)-Pip-Arg^{P}(OPh)₂, Ac-hCha-Cys(MeBzl)-Pip-Arg^{P}(OPh)₂, Cy3-6-Ahx-DPro-hArg-1-Nal-Arg^{P}(OPh)₂, Ac-DPro-hArg-1-Nal-Arg^{P}(OPh)₂, Bodipy-PEG (4)-Tyr (2,6Cl₂—Z)-Nle-Glu(Bzl)-Arg^{P}(OPh)₂ and Ac-Tyr (2,6Cl₂—Z)-Nle-Glu(Bzl)-Arg^{P}(OPh)₂ have been shown on FIGS. 1-8.

### Example 2. Determination of Inhibition Kinetics (K_{obs}/l) for Fluorescently-Labeled ABPs and Inhibitors

The K_{obs}/l parameters towards APC, fIIa, fXa, and fXIa were measured under pseudo first-order conditions (Table 1). The assay buffer contained 20 mM Tris-base, 150 mM NaCl, 5 mM CaCl₂), pH 7.4. Each ABP or inhibitor was serially diluted until the seventh well to obtain the concentrations ranging from 308 nM to 100 UM, depending on the ABP/inhibitor used. The assay conditions were as follows: 20 μL of selected substrate (154 μM SMA5 for APC, 100 μM SMA4 for fIIa, 50 μM SMII18 for fXa, and 100 μM SMX1 for fXIa) was added into the wells containing 20 μL of seven different ABP/inhibitor concentrations. Next, 60 μL of APC, fIIa, fXa, or fXIa (at the concentration of 10 nM) preincubated for 15 min at 37° C. was added, and the fluorescence increase over time was measured (λₑₓ=355 nm, λₑₘ=460 nm) for 30 min. The K_{obs}/l values were calculated using GraphPad Prism and Microsoft Excel software as described previously [26].

TABLE 1
Kinetic parameters (k_{obs}/I) of fluorescent ABPs and inhibitors determined for APC, fIIa, fXa, and fXIa.

| | | k_{obs}/I [M⁻¹s⁻¹] | | | |
|---|---|---|---|---|---|
| | compound | APC | fIIa | fXa | fXIa |
| P-SMA172 | Cy5-6-Ahx-Lys-Dab(Z)-Lys-Arg^{P}(OPh)₂ | 2,250 ± 170 | | | |
| I-SMA17 | Ac-Lys-Dab(Z)-Lys-Arg^{P}(OPh)₂ | 1,980 ± 20 | | | |
| P-SMII222 | Cy7-6-Ahx-hCha-Cys(MeBzl)-Pip-Arg^{P}(OPh)₂ | | 3,500 ± 300 | | |
| I-SMII22 | Ac-hCha-Cys(MeBzl)-Pip-Arg^{P}(OPh)₂ | | 209,000 ± 13,800 | | |
| P-SMX152 | Cy3-6-Ahx-DPro-hArg-1-Nal-Arg^{P}(OPh)₂ | | | 1,800 ± 100 | |
| I-SMX15 | Ac-DPro-hArg-1-Nal-Arg^{P}(OPh)₂ | | | 1,200 ± 100 | |
| P-SMXI52 | Bodipy-PEG(4)-Tyr(2,6Cl₂-Z)-Nle-Glu(Bzl)-Arg^{P}(OPh)₂ | | | | 3,120 ± 100 |
| I-SMXI5 | Ac-Tyr(2,6Cl₂-Z)-Nle-Glu(Bzl)-Arg^{P}(OPh)₂ | | | | 54,700 ± 2,800 |

### Example 3. Coagulation Factors Labeling by ABPs Based on SDS-PAGE Analysis

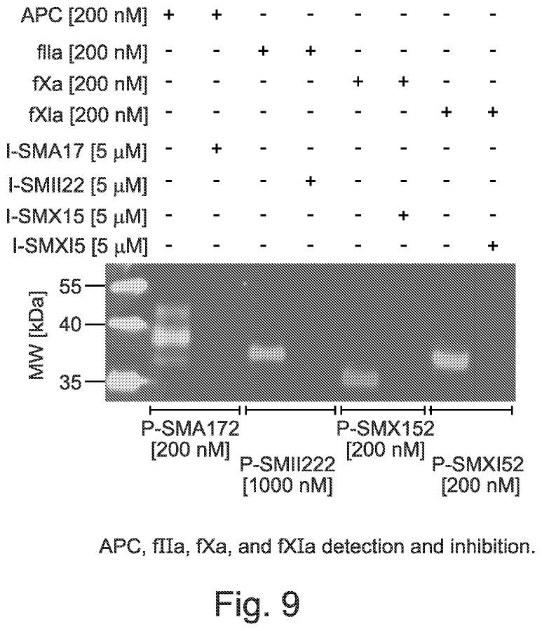

To determine fluorescent ABPs utility, four purified coagulation factors (APC, fIIa, fXa, fXIa) with the constant concentration of 200 nM were incubated with dedicated probe (the probe: enzyme ratio was 1 or 5, depending on the ABP used) in assay buffer (20 mM Tris-base, 150 mM NaCl, 5 mM CaCl₂), pH 7.4) for 30 min at 37° C. P-SMA172, P-SMII222, P-SMX152, and P-SMX152 were used for APC, fIIa, fXa, and fXIa detection, respectively. Each enzyme was incubated with its probe in a volume of 40 μL, followed by the reduction with 20 μL of 3×SDS/DTT for 5 min at 95° C. When testing the utility of the inhibitors, 200 nM of each enzyme was incubated with appropriate inhibitor (final inhibitor concentration 5 M) for 60 min, prior to probe addition (the probe:enzyme ratio was 1 or 5, depending on the ABP used). I-SMA17, I-SMII22, I-SMX15, and I-SMX15 were used for APC, fIIa, fXa, and fXIa inhibition, respectively. The first well was loaded with 0.5 μL of the protein marker PageRuler Prestained Protein Ladder (Thermo Scientific), then 10 μL of each sample was run onto a 12% (w/v) 15-well gel. The SDS-PAGE separation was performed at 200 V for 39 min, followed by transfer to a nitrocellulose membrane (0.2 μm, Bio-Rad) at 10 V for 60 min. The membrane was blocked with 2.5% BSA in TBS-T (Tris-buffered saline with 0.1% (v/v) Tween 20) for 60 min at room temperature. Next, the labeled proteins were directly imaged with lasers of 520 nm for Cy3, 658 nm for Cy5, 784 nm for Cy7, and 488 nm for Bodipy FL using an Azure Biosystems Sapphire Biomolecular Imager and Azure Spot Analysis Software (FIG. 9).

### Example 4. Coagulation Factors Labeling by ABPs in Human Plasma

To evaluate ABPs selectivity and utility APC, fIIa, fXa, and fXIa, a labelling in human plasma was performed. Human plasma was isolated from whole blood using Polymorphprep (Fisher Scientific) and Ficoll Paque Plus (Sigma-Aldrich). 1 mL of Ficoll Paque Plus was layered over 4 mL of Polymorphprep in 15-mL falcon tube, followed by 5 mL of whole blood layer and centrifuged at 450×g for 35 min at room temperature. The obtained plasma was then carefully removed from the cell pellet using a Pasteur pipette and diluted 7.5 times in assay buffer (20 mM Tris-base, 150 mM NaCl, 5 mM CaCl₂), pH 7.4).

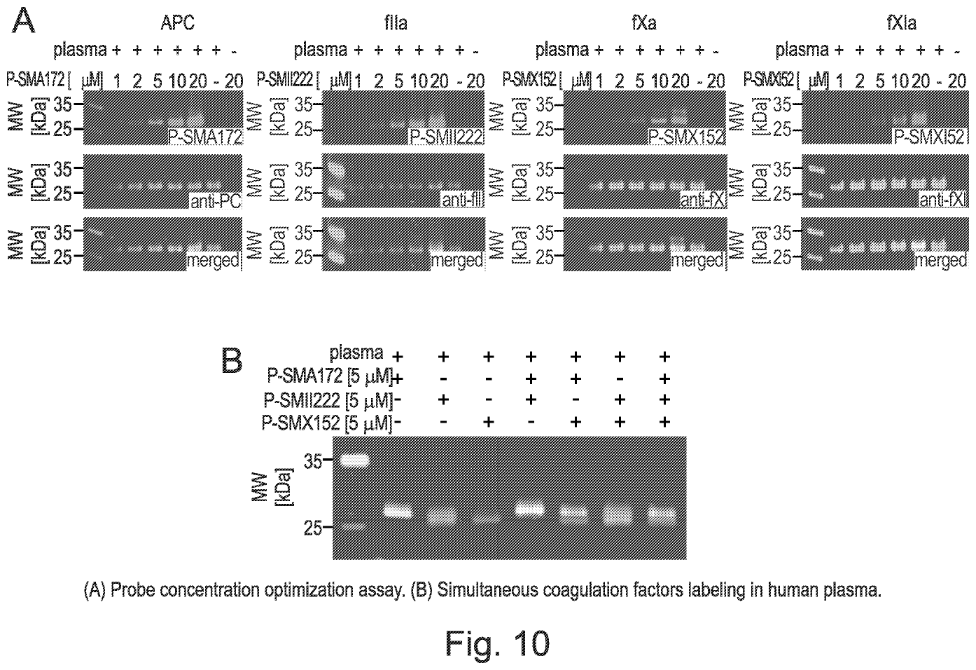

In probe concentration optimization assay (FIG. 10A), human plasma was incubated with each fluorescently-labeled probe separately at varying probe concentrations ranging from 1 to 20 μM. P-SMA172, P-SMII222, P-SMX152, and P-SMXI52 were used for APC, fIIa, fXa, and fXIa detection, respectively. Incubation was carried out in assay buffer (20 mM Tris-base, 150 mM NaCl, 5 mM CaCl₂), pH 7.4) for 60 min at 37° C. Plasma was incubated with probe in a total volume of 40 μL (20 μL of plasma and 20 μL of probe), followed by the reduction with 20 μL of 3×SDS/DTT for 5 min at 95° C. The first well was loaded with 0.5 μL of the protein marker PageRuler Prestained Protein Ladder (Thermo Scientific), then 5 μL of each sample was run onto a 12% (w/v) 15-well gel. The SDS-PAGE separation was performed at 200 V for 39 min, followed by transfer to a nitrocellulose membrane (0.2 μm, Bio-Rad) at 10 V for 60 min. The membrane was blocked with 2.5% BSA in TBS-T (Tris-buffered saline with 0.1% (v/v) Tween 20) for 60 min at room temperature. For APC detection, the membrane was treated with chicken anti-human polyclonal PC antibody (Antibodies-online GmbH, ABIN597333, 1:1,000) for 7 h, followed by incubation with Alexa Fluor 488 goat anti-chicken secondary antibody (Life Technologies, A11039, 2:10,000) for 1 h (both at room temperature). For fIIa detection, the membrane was treated with sheep anti-human polyclonal fII antibody (Haematologic Technologies Inc., PAHFII-S, 1:1,000) for 7 h, followed by incubation with Alexa Fluor 680 donkey anti-sheep secondary antibody (Life Technologies, A21102, 2:10,000) for 1 h (both at room temperature). For fXa detection, the membrane was treated with sheep anti-human polyclonal fX antibody (Haematologic Technologies Inc., PAHFX-S, 1:1,000) for 7 h, followed by incubation with Alexa Fluor 680 donkey anti-sheep secondary antibody (Life Technologies, A21102, 2:10,000) for 1 h (both at room temperature). For fXIa detection, the membrane was treated with sheep anti-human polyclonal fXI antibody (Haematologic Technologies Inc., PAHFXI-S, 1:1,000) for 7 h, followed by incubation with Alexa Fluor 680 donkey anti-sheep secondary antibody (Life Technologies, A21102, 2:10,000) for 1 h (both at room temperature). The membranes were then scanned using an Azure Biosystems Sapphire Biomolecular Imager and Azure Spot Analysis Software as follows: for APC at 658 nm (for Cy5 detection) and 488 nm (for antibody detection), for fIIa at 784 nm (for Cy7 detection) and 658 nm (for antibody detection), for fXa at 520 nm (for Cy3 detection) and 658 nm (for antibody detection), and for fXIa at 488 nm (for Bodipy detection) and 658 nm (for antibody detection).

In simultaneous coagulation factors labeling (FIG. 10B), human plasma was incubated with 5 UM of each fluorescently-labeled probe: P-SMA172, P-SMII222, and P-SMX152. Incubation was carried out in the same assay buffer as used above (60 min, 37° C.). Plasma was incubated with probe in a total volume of 40 L (20 μL of plasma and 20 μL of probes mixture), followed by the reduction with 20 μL of 3×SDS/DTT for 5 min at 95° C. The first well was loaded with 2 μL of the protein marker PageRuler Prestained Protein Ladder (Thermo Scientific), then 5 μL of each sample was run onto a 10% MES (w/v) 15-well gel. The SDS-PAGE separation was performed at 100 V for 140 min. The gel was then directly scanned at 520 nm for Cy3, 658 nm for Cy5, and 784 nm for Cy7 detection using an Azure Biosystems Sapphire Biomolecular Imager and Azure Spot Analysis Software.

### Example 5-Substrates for APC

The following compounds were synthesised according to the procedures outlined in Modrzycka et al. Chemical Science, 2022, 13, 6813-6829 (and the supporting information):
Ac-[L-Lys]-[L-Dab(Z)]-[L-IgI]-[L-Arg]-ACC (SMA5);
Ac-[L-Lys]-[L-Agp]-[L-IgI]-[L-Arg]-ACC (SMA6);
Ac-[L-Lys]-[L-Glu(Bzl)]-[L-IgI]-[L-Arg]-ACC (SMA7);
Ac-[L-Lys]-[L-Dab(Z)]-[L-Val]-[L-Arg]-ACC (SMA9);
Ac-[L-Lys]-[L-Agp]-[L-Val]-[L-Arg]-ACC (SMA10);
Ac-[L-Lys]-[L-Glu(Bzl)]-[L-Val]-[L-Arg]-ACC (SMA11);
Ac-[L-Lys]-[L-Dab(Z)]-[L-Lys]-[L-Arg]-ACC (SMA17);
Ac-[L-Lys]-[L-Glu(Bzl)]-[L-Lys]-[L-Arg]-ACC (SMA19);
Ac-[L-Lys]-[L-Glu(All)]-[L-IgI]-[L-Arg]-ACC (SMA25);
[Ac is acetyl and ACC is 7-amino-4-carbamoylmethylcoumarin].
   Determination of the Kinetic Parameters (K_{cat}/K_{M}) for the Substrates SMA5, SMA6 and SMA17s

The K_{cat}/K_{M} parameters towards APC, thrombin and fXa were measured using the procedure described in Modrzycka et al. Chemical Science, 2022, 13, 6813-6829.

The results obtained are shown below:

TABLE 1
The kinetic parameters (k_{cat}/K_{M}) of the most selective substrates for
the investigated of coagulation factors. The data represent the mean
values ± s.d.; n = 3, where n is the number of independent experiments.

| | | k_{cat}/K_{M} [M⁻¹s⁻¹] | | |
|---|---|---|---|---|
| | substrate | APC | thrombin | fXa |
| SMA 5 | Ac-Lys-Dab(Z)-Igl-Arg-ACC | 71,000 ± 2,000 | 1,800 ± 80 | 4,000 ± 150 |
| SMA 6 | Ac-Lys-Agp-Igl-Arg-ACC | 40,000 ± 1,600 | 180 ± 7 | 1,300 ± 90 |
| SMA 17 | Ac-Lys-Dab(Z)-Lys-Arg-ACC | 10,000 ± 600 | 130 ± 8 | 190 ± 6 |
| SMA 26 | Ac-Lys-βhLys-Igl-Arg-ACC | 15,500 ± 300 | 60 ± 2 | 380 ± 13 |

### APC Substrate Design and Validation

Out of a screen of twenty-five APC tetrapeptide fluorogenic substrates (named SMA1-SMA26) described in Modrzycka et al. Chemical Science, 2022, 13, 6813-6829, the substrates SMA5, SMA6, and SMA17 showed the most activity and selectivity for APC.

The screen showed that P2 (corresponding to A2 in Formula X) was crucial in terms of discriminating APC from thrombin and fXa since L-IgI and L-Lys were the most selective amino acids for APC and were poorly recognized by the other coagulation factors. It was also noted that exchanging the long L-Nle(O-Bzl)(SMA4) at P3 (corresponding to A₃ in Formula X) for the shorter L-Dab(Z) (SMA1), L-Agp (SMA2) and L-Glu(Bzl) (SMA3) resulted in significant increases in activity (13-, 22- and 5.5-fold, respectively). Compared to the best natural substrate (SMA54), the best substrate that contained unnatural amino acids (SMA5) was cleaved approximately 50-fold more efficiently, suggesting that the use of the HyCoSuL approach allowed us to obtain active and selective chemical tools for APC. To minimize potential cross reactivity, a detailed kinetic analysis of the four APC-selective substrates was performed against the other proteases. The catalytic efficiency was determined and revealed that the most potent substrate, SMA5 (Ac-Lys-Dab(Z)-IgI-Arg-ACC, K_{cat}/K_{M}=71,000±2,000 M⁻¹s⁻¹), was also recognized by thrombin (K_{cat}/K_{M}=1,800±80 M⁻¹s⁻¹) and fXa (K_{cat}/K_{M}=4,000±150 M⁻¹s⁻¹). SMA17 (Ac-Lys-Dab(Z)-Lys-Arg-ACC, K_{cat}/K_{M}=10,000±600 M⁻¹s⁻¹) displayed a very high K_{cat}/K_{M} selectivity ratio and almost nondetectable hydrolysis when tested with other coagulation factors.

### REFERENCES

1. Furie, B. and B. C. Furie, The molecular basis of blood coagulation. Cell, 1988. 53 (4): p. 505-18.
2. Chaudhry, R., S. M. Usama, and H. M. Babiker, Physiology, Coagulation Pathways, in StatPearls. 2021: Treasure Island (FL).
3. Ferland, G., The vitamin K-dependent proteins: an update. Nutr Rev, 1998. 56 (8): p. 223-30.
4. Suttie, J. W., Synthesis of vitamin K-dependent proteins. FASEB J, 1993. 7 (5): p. 445-52.
5. Harris, J. L., et al., Rapid and general profiling of protease specificity by using combinatorial fluorogenic substrate libraries. Proc Natl Acad Sci USA, 2000. 97 (14): p. 7754-9.
6. Wendelboe, A. M. and G. E. Raskob, Global Burden of Thrombosis: Epidemiologic Aspects. Circ Res, 2016. 118 (9): p. 1340-7.
7. Raskob, G. E., et al., Thrombosis: a major contributor to global disease burden. Arterioscler Thromb Vasc Biol, 2014. 34 (11): p. 2363-71.
8. Negrier, C., M. Shima, and M. Hoffman, The central role of thrombin in bleeding disorders. Blood Rev, 2019. 38: p. 100582.
9. Polderdijk, S. G. I., T. P. Baglin, and J. A. Huntington, Targeting activated protein C to treat hemophilia. Curr Opin Hematol, 2017. 24 (5): p. 446-452.
10. Rosendaal, F. R., Venous thrombosis: a multicausal disease. Lancet, 1999. 353 (9159): p. 1167-73.
11. Shen, G. X., Inhibition of thrombin: relevance to anti-thrombosis strategy. Front Biosci, 2006. 11: p. 113-20.
12. Meijers, J. C., et al., High levels of coagulation factor XI as a risk factor for venous thrombosis. N Engl J Med, 2000. 342 (10): p. 696-701.
13. Cushman, M., et al., Coagulation factors IX through XIII and the risk of future venous thrombosis: the Longitudinal Investigation of Thromboembolism Etiology. Blood, 2009. 114 (14): p. 2878-2883.
14. Levi, M. and H. Ten Cate, Disseminated intravascular coagulation. N Engl J Med, 1999. 341 (8): p. 586-92.
15. Lowenberg, E. C., et al., Coagulation factor XI as a novel target for antithrombotic treatment. J Thromb Haemost, 2010. 8 (11): p. 2349-57.
16. Yang, D. T., et al., Elevated factor XI activity levels are associated with an increased odds ratio for cerebrovascular events. Am J Clin Pathol, 2006. 126 (3): p. 411-5.
17. Doggen, C. J., F. R. Rosendaal, and J. C. Meijers, Levels of intrinsic coagulation factors and the risk of myocardial infarction among men: Opposite and synergistic effects of factors XI and XII. Blood, 2006. 108 (13): p. 4045-51.
18. Cantrell, R. and J. S. Palumbo, The thrombin-inflammation axis in cancer progression. Thromb Res, 2020. 191 Suppl 1: p. S117-S122.
19. Akiyama, H., et al., Thrombin accumulation in brains of patients with Alzheimer's disease. Neurosci Lett, 1992. 146 (2): p. 152-4.
20. Iannucci, J., W. Renehan, and P. Grammas, Thrombin, a Mediator of Coagulation, Inflammation, and Neurotoxicity at the Neurovascular Interface: Implications for Alzheimer's Disease. Front Neurosci, 2020. 14: p. 762.
21. Simmons, J. and J. F. Pittet, The coagulopathy of acute sepsis. Curr Opin Anaesthesiol, 2015. 28 (2): p. 227-36.
22. Jordan, K. R., et al., Thrombin generation and activity in multiple sclerosis. Metab Brain Dis, 2021. 36 (3): p. 407-420.
23. Aliter, K. F. and R. A. Al-Horani, Thrombin Inhibition by Argatroban: Potential Therapeutic Benefits in COVID-19. Cardiovasc Drugs Ther, 2021. 35 (2): p. 195-203.
24. Al-Horani, R. A., Potential Therapeutic Roles for Direct Factor Xa Inhibitors in Coronavirus Infections. Am J Cardiovasc Drugs, 2020. 20 (6): p. 525-533.
25. Sivaraman, A., et al., Synthesis and Structure-Activity Relationships of Arylsulfonamides as AIMP2-DX2 Inhibitors for the Development of a Novel Anticancer Therapy. J Med Chem, 2020. 63 (10): p. 5139-5158.
26. Kasperkiewicz, P., et al., Design of ultrasensitive probes for human neutrophil elastase through hybrid combinatorial substrate library profiling. Proc Natl Acad Sci USA, 2014. 111 (7): p. 2518-23.

1. A compound represented by the formula X shown below, or a pharmaceutically acceptable salt thereof:
Zₓ-A₄-A₃-A₂-A₁-R₁ₓ formula X
wherein:
Zₓ is hydrogen, an amino terminal capping group (e.g. (2-6C)alkanoyl, such as acetyl (Ac) or propionyl) or group of formula-R₂ₓ-R₃ₓ
wherein:
R₂ₓ is a linker group; and
R₃ₓ is a fluorescent group or biotin;
R₁ₓ is a reactive binding group or a detectable leaving group;
A₁ is L-arginine (L-Arg);
A₂ is selected from L-lysine (L-Lys), L-piperidine-2-carboxylic acid (L-Pip), L-3-(1-naphthyl)-L-alanine (L-1-Nal), L-glutamic-acid-gamma-benzyl ester (L-Glu(Bzl)), L-glutamine (L-Gln), L-leucine (L-Leu), L-norleucine (L-Nle), L-threonine (L-Thr), L-valine (L-Val), L-homoarginine (L-hArg), 4-amino-L-phenylalanine (L-Phe (4-NH₂)), L-homoalanine (L-Abu), L-phenylglycine (L-Phg), 2-indanyl-L-glycine (L-IgI), L-neopentyl-glycine (L-NptGly), or L-norvaline (L-Nva);
A₃ is selected from benzyloxycarbonyl-L-2,4-diaminobutyric acid (L-Dab(Z)), 4-methylbenzyl-L-cysteine (L-Cys(MeBzl)), L-homoarginine (L-hArg), L-norleucine (L-Nle), D-Arginine (D-Arg), D-homophenylalanine (D-hPhe), guanidino-L-alanine (L-Agp), L-glutamic acid benzyl ester (LGlu(Bzl)), L-glutamic acid allyl ester (LGlu(All)), L-methionine sulfone (L-Met (O) 2), or L-biphenylalanine (L-Bip);
A₄ is selected from L-lysine (L-Lys), L-homocyclohexylalanine (L-hCha), D-proline (D-Pro), 2,6-dichlorobenzyl-L-tyrosine (L-Tyr (2,6-Cl₂—Z), L-arginine (L-Arg), L-tryptophan (L-Trp), amino-L-homoalanine (L-Dab), N-trifluoroacetyl-L-lysine (L-Lys (TFA)), N-2-chlorobenzyloxycarbonyl-L-lysine (L-Lys (2-Cl—Z)), L-homoarginine (L-hArg), 4-amino-L-phenylalanine (L-Phe (4-NH₂)), 4-iodo-L-phenylalanine (L-Phe (4-I)), 3-benzothienyl-L-alanine (L-Bta), or L-biphenylalanine (L-Bip).

2. A compound according to claim 1, wherein
A₂ is selected from L-lysine (L-Lys), L-glutamine (L-GIn), L-leucine (L-Leu), L-norleucine (L-Nle), L-threonine (L-Thr), L-valine (L-Val), L-homoarginine (L-hArg), 4-amino-L-phenylalanine (L-Phe (4-NH₂)), L-homoalanine (L-Abu), L-phenylglycine (L-Phg), 2-indanyl-L-glycine (L-IgI), L-neopentyl-glycine (L-NptGly), or L-norvaline (L-Nva);
A₃ is selected from benzyloxycarbonyl-L-2,4-diaminobutyric acid (L-Dab(Z)), D-Arginine (D-Arg), D-homophenylalanine (D-hPhe), guanidino-L-alanine (L-Agp), L-glutamic acid benzyl ester (LGlu(Bzl)), L-glutamic acid allyl ester (LGlu(All)), L-methionine sulfone (L-Met (O) 2), or L-biphenylalanine (L-Bip);
A₄ is selected from L-lysine (L-Lys), L-arginine (L-Arg), L-tryptophan (L-Trp), amino-L-homoalanine (L-Dab), N-trifluoroacetyl-L-lysine (L-Lys(TFA)), N-2-chlorobenzyloxycarbonyl-L-lysine (L-Lys (2-Cl—Z)), L-homoarginine (L-hArg), 4-amino-L-phenylalanine (L-Phe (4-NH₂)), 4-iodo-L-phenylalanine (L-Phe (4-I)), 3-benzothienyl-L-alanine (L-Bta), or L-biphenylalanine (L-Bip).

3. A compound according to claim 1 or claim 2, wherein A₂ is selected from one of the following options:
(i) L-lysine (L-Lys),
L-valine (L-Val), or
2-indanyl-L-glycine (L-IgI);
(ii) L-lysine (L-Lys), or
2-indanyl-L-glycine (L-IgI);
(iii) L-lysine (L-Lys);
(iv) L-valine (L-Val);
(v) 2-indanyl-L-glycine (L-IgI).

4. A compound according to any one of the preceding claims, wherein A₃ is selected from one of the following options:
(i) benzyloxycarbonyl-L-2,4-diaminobutyric acid (L-Dab(Z)),
guanidino-L-alanine (L-Agp),
L-glutamic acid benzyl ester (LGlu(Bzl)), or
L-glutamic acid allyl ester (LGlu(All));
(ii) benzyloxycarbonyl-L-2,4-diaminobutyric acid (L-Dab(Z)), or
guanidino-L-alanine (L-Agp);
(iii) benzyloxycarbonyl-L-2,4-diaminobutyric acid (L-Dab(Z));
(iv) guanidino-L-alanine (L-Agp).

5. A compound according to any one of the preceding claims, wherein A₄ is selected from one of the following options:
(i) L-lysine (L-Lys),
L-arginine (L-Arg),
L-tryptophan (L-Trp), or
amino-L-homoalanine (L-Dab);
(ii) L-lysine (L-Lys).

6. A compound according to any one of the preceding claims, wherein the compound is selected from any one of the following, or a pharmaceutically acceptable salt thereof:
Zₓ-[L-Lys]-[L-Dab(Z)]-[L-IgI]-[L-Arg]-R₁ₓ
Zₓ-[L-Lys]-[L-Agp]-[L-IgI]-[L-Arg]-R₁ₓ
Zₓ-[L-Lys]-[L-Glu(Bzl)]-[L-IgI]-[L-Arg]-R₁ₓ
Zₓ-[L-Lys]-[L-Dab(Z)]-[L-Val]-[L-Arg]-R₁ₓ
Zₓ-[L-Lys]-[L-Agp]-[L-Val]-[L-Arg]-R₁ₓ
Zₓ-[L-Lys]-[L-Glu(Bzl)]-[L-Val]-[L-Arg]-R₁ₓ
Zₓ-[L-Lys]-[L-Dab(Z)]-[L-Lys]-[L-Arg]-R₁ₓ
Zₓ-[L-Lys]-[L-Glu(Bzl)]-[L-Lys]-[L-Arg]-R₁ₓ
Zₓ-[L-Lys]-[L-Glu(All)]-[L-IgI]-[L-Arg]-R₁ₓ;
wherein Zₓ and R₁ₓ are both as defined in claim 1.

7. A compound according to any one of the preceding claims, wherein the compound is selected from any one of the following, or a pharmaceutically acceptable salt thereof:
Ac-[L-Lys]-[L-Dab(Z)]-[L-IgI]-[L-Arg]-ACC (SMA5);
Ac-[L-Lys]-[L-Agp]-[L-IgI]-[L-Arg]-ACC (SMA6);
Ac-[L-Lys]-[L-Glu(Bzl)]-[L-IgI]-[L-Arg]-ACC (SMA7);
Ac-[L-Lys]-[L-Dab(Z)]-[L-Val]-[L-Arg]-ACC (SMA9);
Ac-[L-Lys]-[L-Agp]-[L-Val]-[L-Arg]-ACC (SMA10);
Ac-[L-Lys]-[L-Glu(Bzl)]-[L-Val]-[L-Arg]-ACC (SMA11);
Ac-[L-Lys]-[L-Dab(Z)]-[L-Lys]-[L-Arg]-ACC (SMA17);
Ac-[L-Lys]-[L-Glu(Bzl)]-[L-Lys]-[L-Arg]-ACC (SMA19);
Ac-[L-Lys]-[L-Glu(All)]-[L-IgI]-[L-Arg]-ACC (SMA25);
wherein Ac is acetyl and ACC is 7-amino-4-carbamoylmethylcoumarin.

8. A compound represented by the following chemical formula 1: 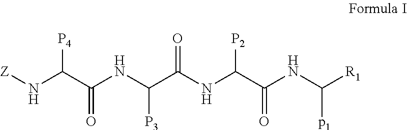
wherein:
Z is acetyl (Ac) or group of formula R₃-R₂-
wherein: R₂ is linker group such as: PEG or 6-aminohexanoic acid; and
R₃ is fluorescent group such as: 1-(5-carboxypentyl)-3,3-dimethyl-2-((1E,3Z)-3-(1,3,3-trimethylindolin-2-ylidene) prop-1-enyl)-3H-indolium (Cyanine 3), 1-(5-carboxypentyl)-2-((1E,3E,5E)-5-(1,3-dimethylindolin-2-ylidene) penta-1,3-dienyl)-3,3-dimethyl-3H-indolium (Cyanine 5), 1-(5-carboxypentyl)-3,3-dimethyl-2-((E)-2-((E)-3-((Z)-2-(1,3,3-trimethylindolin-2-ylidene)ethylidene)cyclohex-1-enyl) vinyl)-3H-indolium (Cyanine 7), or 4,4-difluoro-5,7-dimethyl-4-bora-3a,4a-diaza-s-indacene-3-propionic acid (Bodipy)
R₁ is reactive binding group such as: diphenyl phosphonate and its derivatives
P₁ is functional group: 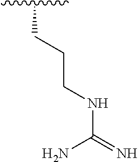
L-Arginine (L-Arg) side chain;
P₂ is a functional group selected from: 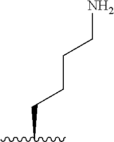
L-Lysine (L-Lys) side chain, 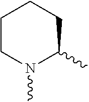
L-piperidine-2-carboxylic acid (L-Pip) side chain, 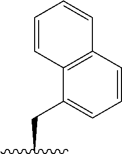
3-(1-naphthyl)-L-alanine (L-1-Nal) side chain, 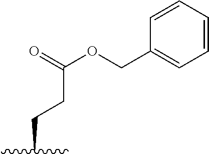
L-glutamic-acid-gamma-benzyl ester (L-Glu(Bzl)) side chain,
P₃ is functional group selected from: 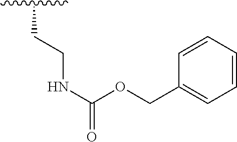
benzyloxycarbonyl-L-2,4-diaminobutyric acid (L-Dab(Z)) side chain, 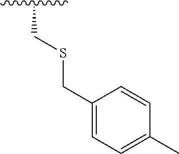
4-methylbenzyl-L-cysteine (L-Cys(MeBzl)) side chain, 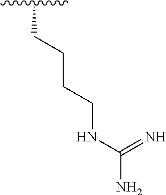
L-homoarginine (L-hArg) side chain, 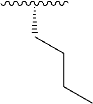
L-norleucine (L-Nle) side chain,
P₄ is functional group selected from: 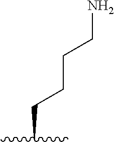
L-Lysine (L-Lys) side chain, 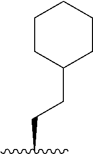
L-homocyclohexylalanine (L-hCha) side chain, 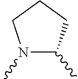
D-proline (D-Pro) side chain, 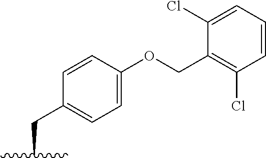
2,6-dichlorobenzyl-L-tyrosine (L-Tyr (2,6-Clz-Z) side chain.

9. A compound according to claim 8, characterized in that the compound is represented by the following chemical formulas 2-5: 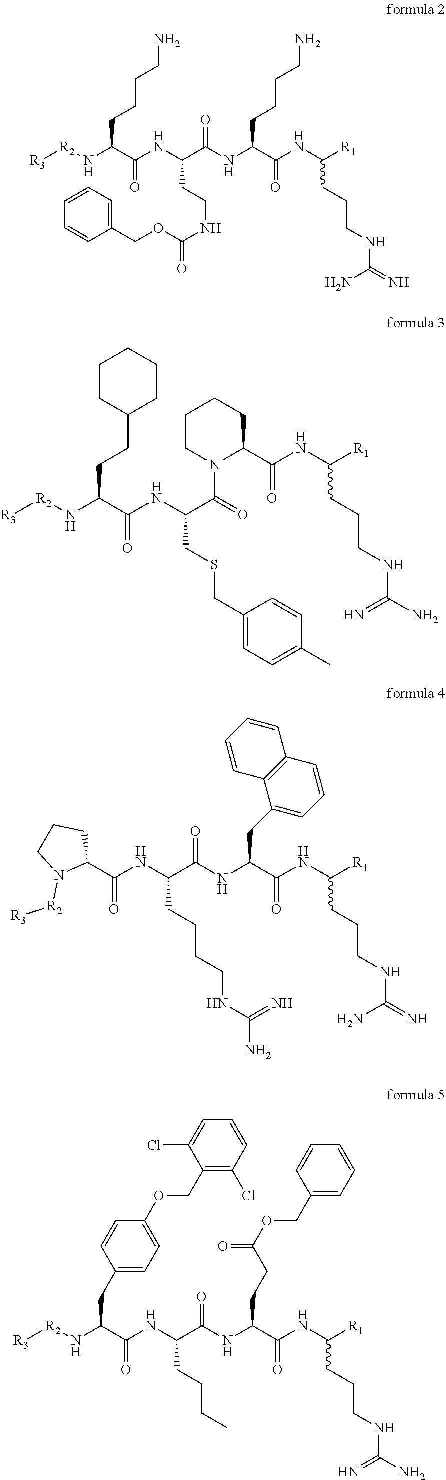
wherein:
R₃ is acetyl (Ac) or fluorescent group such as: 1-(5-carboxypentyl)-3,3-dimethyl-2-((1E,3Z)-3-(1,3,3-trimethylindolin-2-ylidene) prop-1-enyl)-3H-indolium (Cyanine 3), 1-(5-carboxypentyl)-2-((1E,3E,5E)-5-(1,3-dimethylindolin-2-ylidene) penta-1,3-dienyl)-3,3-dimethyl-3H-indolium (Cyanine 5), 1-(5-carboxypentyl)-3,3-dimethyl-2-((E)-2-((E)-3-((Z)-2-(1,3,3-trimethylindolin-2-ylidene)ethylidene)cyclohex-1-enyl) vinyl)-3H-indolium (Cyanine 7), or 4,4-difluoro-5,7-dimethyl-4-bora-3a,4a-diaza-s-indacene-3-propionic acid (Bodipy), R₂ is linker group such as: PEG or 6-aminohexanoic acid, R₁ is reactive binding group such as: diphenyl phosphonate and its derivatives.

10. A compound according to claim 8 or 9, wherein the compound is selected from one of the following:
Cy5-6-Ahx-Lys-Dab(Z)-Lys-Arg^{P}(OPh)₂,
Ac-Lys-Dab(Z)-Lys-Arg^{P}(OPh)₂,
Cy7-6-Ahx-hCha-Cys(MeBzl)-Pip-Arg^{P}(OPh)₂,
Ac-hCha-Cys(MeBzl)-Pip-Arg^{P}(OPh)₂,
Cy3-6-Ahx-DPro-hArg-1-Nal-Arg^{P}(OPh)₂,
Ac-DPro-hArg-1-Nal-Arg^{P}(OPh)₂,
Bodipy-PEG (4)-Tyr (2,6Cl₂—Z)-Nle-Glu(Bzl)-Arg^{P}(OPh)₂ and
Ac-Tyr(2,6Cl₂—Z)-Nlw-Glu(Bzl)-Arg^{P}(OPh)₂.

11. Use of a compound, or a salt thereof, according to any one of the preceding claims for the detection or inhibition ex-vivo of blood coagulation protease, wherein said compound is inhibitor of one or more coagulation proteases selected from APC, fIIa, fXa and fXIa.

12. A compound according to any one of claims 1 to 10, or a salt thereof, for use in the inhibition of blood coagulation wherein said compound is acting as inhibitor of one or more coagulation proteases selected from fIIa, fXa and fXIa.

13. A compound according to any one of claims 1 to 10, or a salt thereof, for use in treatment or prevention of an illness connected with, or associated with, coagulation protease activity, wherein said compound is acting as inhibitor of one or more coagulation proteases selected from APC, fIIa, fXa and fXIa.

14. A compound according to any one of claims 1 to 10, or a salt thereof, for use according to claim 13, wherein said illness has been selected from the group consisting of: bleeding disorders (e.g. hemophilia), thrombosis, disseminated intravascular coagulation, ischemic stroke, myocardial infarction, cancer, Alzheimer's disease, sepsis, multiple sclerosis and COVID-19.

15. Use of a compound according to claim 11, or a compound for use according to claim 13, wherein said compound is acting as inhibitor of APC and is represented by the chemical formula 2 as defined in claim 9.

16. Use of a compound according to claim 11, or a compound for use according to claim 12 or 13, wherein said compound is acting as inhibitor of fIIa and is represented by the chemical formula 3 as defined in claim 9.

17. Use of a compound according to claim 11, or a compound for use according to claim 12 or 13, wherein said compound is acting as inhibitor of fXa and is represented by the chemical formula 4 as defined in claim 9.

18. Use of a compound according to claim 11, or a compound for use according to claim 12 or 13, wherein said compound is acting as an inhibitor of fXIa and is represented by the chemical formula 5 as defined in claim 9.
